(19) United States
(12) Patent Application Publication
(10) Pub. No.: US 2003/0118649 A1
(43) Pub. Date: Jun. 26, 2003

(54) Drug delivery devices and methods

(76) Inventors: Jinming Gao, Pepper Pike, OH (US); Feng Qian, Cleveland, OH (US); Agata Exner, Cleveland Heights, OH (US); John R. Haaga, Chagrin Falls, OH (US)

Correspondence Address: ROPES & GRAY, ONE INTERNATIONAL PLACE, BOSTON, MA, 02110-2624, US

(21) Appl. No.: 10/265,355

(22) Filed: Oct. 4, 2002

Related U.S. Application Data
(60) Provisional application No. 60/374,643, filed on Apr. 23, 2002.
(60) Provisional application No. 60/326,939, filed on Oct. 4, 2001.

(51) Int. Cl. A61K 9/24

(52) U.S. Cl. 424/471

(57) ABSTRACT

The present invention relates to drug delivery devices that provide sustained release of a therapeutic agent upon implantation into a patient. In certain embodiments, the devices provide an initial burst release of an agent, followed by sustained release of that agent, or of a different agent. The devices comprise a central core surrounded at least one membrane or coating.

This application is based on U.S. Provisional Applications No. 60/374,643, filed Apr. 23, 2002, and No. 60/326,939, filed Oct. 4, 2001, the specifications of which are hereby incorporated by reference in their entirety.

### BACKGROUND

Over the years, various drugs have been developed to assist in the treatment of a wide variety of ailments and diseases. Due to the risks that certain drugs impose, researchers have developed systems for administering such drugs to aid in the treatment of these ailments and diseases. Many of these systems provide a release rate which reduces the occurrence of detrimental side effects.

With conventional dosing (tablets, injections, etc.), the concentration of drug in the area being treated increases from an initial ineffective concentration to an effective concentration. Frequently the concentration may actually reach some toxic threshold. After a relatively short period, however, the drug concentration decreases as drug is either metabolized in the body or is eliminated. Eventually, drug levels decrease so low that therapeutic levels are no longer maintained. A second dose is then given and the cycle is repeated. The goal of sustained-release systems is to maintain drug levels within the therapeutic range and ideally a constant level.

In order to achieve constant levels, drugs should be released from a delivery system at a rate that does not change with time (so called zero-order release). Preferably, the initial dose of a drug is the therapeutic dose which is maintained by the delivery system. In many systems, however, the release rate is proportional to time (i.e., “first order”) or the square root of time (or Fickian).

Linear release is achievable with some types of reservoir systems, such as tubes, fibers laminates, or microspheres. In these systems, a drug reservoir is coated in a rate-controlling membrane. Drug diffusion across the membrane is rate limiting and is constant (zero order) as long as the membrane's permeability does not change and as long as the concentration of drug in the reservoir is constant (i.e., as long as there is an excess of drug in the reservoir).

In matrix systems, drug is dispersed throughout a matrix and is released as it dissolves and diffuses through the matrix. A drug is released from the outer surface of the matrix first, this layer becomes depleted, and drug that is released from further within the core of the device must then diffuse through the depleted matrix. The net result is that the release rate slows down and Fickian release is common. With matrix systems, zero-order release is very difficult to achieve. The same principles apply to release from gels.

Another type of device for controlling the administration of such drugs is produced by coating a drug with a polymeric material permeable to the passage of the drug to obtain the desired effect. Such devices are particularly suitable for treating a patient at a specific local area without having to expose the patient's entire body to the drug. This is advantageous because any possible side effects of the drug could be minimized.

The above described systems and devices are intended to provide sustained release of drugs effective in treating patients at a desired local or systemic level for obtaining certain physiological or pharmacological effects. However, there are many disadvantages associated with their use including the fact that it is often times difficult to obtain the desired release rate of the drug. The need for a better release system is especially significant in the treatment of hyperproliferative diseases.

### SUMMARY OF THE INVENTION

The present application provides a multi-layer dual-release drug delivery device which can be used to provide localized, sustained delivery of therapeutic agents, e.g., to treat cancer cells, such as solid tumors. In one aspect, the application provides a multi-layer drug delivery device for cancer cells, such as liver cancer cells, that remain following thermoablation.

In one embodiment, the device is a double-layer polymeric device with dual-release kinetics for long term (preferably over more than 1 week) delivery of a therapeutic agent to a patient. In certain embodiments, the device delivers the agent locally, rather than systemically, for example, to deliver therapeutic, e.g., chemotherapeutic, agents to an area of patient's body, such as the site of ablation of a solid tumor. The double layer, dual-release device provides an initial loading dosage followed by a maintenance dosage to the area. The double-layer, dual-release device is a reservoir-type system wherein the outer layer is a film or membrane containing a drug, such as an anticancer drug, and optionally other water-soluble components, while the inner layer or core is a monolithic mixture of a drug, such as an anticancer drug, and biocompatible polymers or other matrix materials or excipients, preferably ones that can provide fast drug release kinetics. When such a double-layer device is implanted inside the patient's tissue, the drug entrapped in the outer membrane is released immediately to provide an initial loading dosage. At the same time, dissolution of the drug and other water-soluble components renders the outer membrane porous. The porous membrane then controls the subsequent release of drug contained inside the inner core, and sustained release is then achieved. This provides a maintenance dose until all drug entrapped inside the inner core is released. The loading dose of the dual release device depends on the amount of drug contained inside the outer membrane, while the maintenance dosage depends on the drug loading of the inner core. The sustained release time can be adjusted by varying the amount of drug contained inside the inner core and the degradation time and proportion of water soluble components of the outer layer. The sustained release rate is controlled by the porosity, tortuosity, and the membrane thickness of the outer layer. The double-layer dual-release device may be employed for intratumoral drug delivery.

In a further embodiment, the multi-layer drug delivery device comprises three layers. The first layer or core is a monolithic mixture of a therapeutic agent and a biocompatible material such as an excipient. The second layer is a membrane that surrounds the core, is formed from a biocompatible material, and comprises water-soluble components whose dissolution results in the formation of pores in the second layer. In certain embodiments, the second layer further comprises the therapeutic agent which is contained within the core, although it may alternatively or additionally comprise a different therapeutic agent, or none at all. The third layer is a membrane which encases the second layer and the core. The third layer is a film formed from a water soluble material, preferably a water-soluble polymer. The outer layer may further comprise a therapeutic agent. The therapeutic agent(s) located in the core and the outer layer may be the same or different.

Both embodiments of the multi-layer delivery device are useful for local therapy of cancer cells, particularly liver cancer cells that survive thermoablation.

In another aspect, the present invention provides a systemic double-layer drug delivery device. Such double-layer device comprises an inner core which is a monolithic mixture of the agent and a biocompatible polymer or other matrix material and an outer layer comprising a polymeric film that contains water-soluble components. Introduction of the device into the bloodstream or other part of a patient's body results in dissolution of the water-soluble components, formation of pores in the membrane, and sustained release of the agent from the core. The rate of release of the agent from the core is regulated by the porosity, tortuosity, and the thickness of the outer layer. The sustained release time can be adjusted by varying the amount of drug contained inside the inner core and the degradation time of the outer layer. Preferably, the device releases the therapeutic agent(s) in the core over a few hours to a few months.

In preferred embodiments, the polymer(s) included in the device are biodegradable. In certain embodiments, a polymer included in the core degrades more rapidly under physiologic conditions than the outer membrane(s) do.

In another aspect, the present invention provides a mathematical model and method of using the same to develop and optimize the design of present multi-layer delivery system.

In one aspect, the invention provides a biodegradable dual-release drug delivery device, comprising a central core comprising a first bioactive agent, and a first layer disposed around the central core comprising a second bioactive agent and a biodegradable polymer, whereby, upon placement in a biological environment, the second bioactive agent is released, resulting in sustained release of the first bioactive agent. In certain embodiments, release of the second bioactive agent renders the first layer porous. In certain embodiments, the first layer includes a water-soluble component that dissolves upon placement in a biological environment, thereby rendering the first layer porous. In certain embodiments, the water-soluble component comprises a water-soluble polymer. In certain embodiments, the water-soluble component comprises water-soluble inclusions. In certain embodiments, the water-soluble inclusions comprise crystals of a biocompatible salt or sugar. In certain embodiments, the first layer comprises a polymer selected from polylactic acid (PLA) and poly(lactic-glycolic acid) (PLGA).

In certain embodiments, the bioactive agent(s) comprise an analgesic agent, anti-cancer agent, antinflammatory agent, anti-fungal agent, anti-viral agent, cell transport/mobility impending agent, beta-blocker, immunological response modifier, peptide or protein, heat shock protein, steroidal compound, neuroprotectant, antibiotic, antibacterial, antiallergenic, anti-inflammatory, decongestant, miotic and anti-cholinesterase, angiogenesis inhibitor, permeability enhancer, or mydriatic. In certain embodiments, the first and second bioactive agent are the same. In certain embodiments, at least one of the first and second bioactive agents is an anti-cancer agent. In certain embodiments, the first layer comprises from 0.1 to 60% bioactive agent, preferably from 1 to 45%, even more preferably between 5 and 35%. In certain embodiments, the core comprises an excipient. In certain embodiments, the device is a cylindrical millirod. In certain embodiments, the device provides local delivery of the bioactive agent(s), while in others, the device may provide sustained delivery.

In certain embodiments, the device further includes a water-soluble second layer disposed around the first layer, optionally including a third bioactive agent, which may be the same or different from the first and second bioactive agents. In certain embodiments, the second layer comprises a water-soluble polymer.

In certain embodiments, the first bioactive agent is released at a therapeutically effective concentration for at least two days, at least a week, or even at least a month. In certain embodiments, the device achieves a therapeutically effective concentration of the bioactive agent(s) within three days, preferably two or even one day.

In another aspect, the invention provides a biodegradable drug delivery device, having a central core comprising a first bioactive agent, a first layer disposed around the central core, comprising a water-soluble component and a biodegradable polymer, and a water-soluble second layer comprising a second bioactive agent disposed around the first layer, whereby, upon placement in a biological environment, the second bioactive agent is released and the water-soluble component dissolves rendering the first layer porous, thereby resulting in sustained release of the first bioactive agent. In certain embodiments, the device is a cylindrical millirod. In certain embodiments, the core comprises an excipient. In certain embodiments, the first layer comprises a polymer selected from polylactic acid (PLA) and poly(lactic-glycolic acid) (PLGA). In certain embodiments, the water-soluble component comprises a water-soluble polymer. In certain embodiments, the water-soluble component comprises water-soluble inclusions. In certain embodiments, the water-soluble inclusions comprise crystals of a biocompatible salt or sugar. In certain embodiments, the second layer comprises a water-soluble polymer.

In certain embodiments, the first and second bioactive agent are the same. In certain embodiments, at least one of the first and second bioactive agents is an anti-cancer agent. In certain embodiments, the bioactive agent(s) comprise an analgesic agent, anti-cancer agent, antinflammatory agent, anti-fungal agent, anti-viral agent, cell transport/mobility impending agent, beta-blocker, immunological response modifier, peptide or protein, heat shock protein, steroidal compound, neuroprotectant, antibiotic, antibacterial, antiallergenic, anti-inflammatory, decongestant, miotic and anti-cholinesterase, angiogenesis inhibitor, permeability enhancer, or mydriatic.

In certain embodiments, the first bioactive agent is released at a therapeutically effective concentration for at least two days, at least a week, or even at least a month. In certain embodiments, the device achieves a therapeutically effective concentration of the bioactive agent(s) within three days, preferably two or even one day. In certain embodiments, the device provides local delivery of the bioactive agent(s).

In yet another aspect, the invention provides a method of administering a bioactive agent to a patient by implanting into a patient a device as described above.

In certain embodiments, the device comprises an anticancer agent and is placed at the site of a thermoablated tumor. In certain embodiments, wherein the device comprises an analgesic agent and is placed at the site of a wound. In certain embodiments, the device comprises an antibiotic or antifungal agent and is placed at the site of an infection.

In still another aspect, the invention provides a method for manufacturing a biodegradable drug delivery device by providing a central core comprising a first bioactive agent, and disposing a layer around the central core, the layer comprising a second bioactive agent, a water-soluble component, and a biodegradable polymer.

In still another aspect, the invention provides a method for manufacturing a biodegradable drug delivery device by providing a central core comprising a first bioactive agent and an excipient, disposing around the central core a first layer comprising a water-soluble component and a biodegradable polymer, and disposing around the first layer a water-soluble second layer comprising a second bioactive agent.

### BRIEF DESCRIPTION OF THE DRAWINGS

FIG. 1. SEM images of membrane-encased polymer millirods. (a) Millirod before salt leaching; (b) millirod after salt leaching in PBS buffer for 4 hours. The scale bar is 100 μm in both images.

FIG. 2. Release profiles of three different types of millirods. (a) Monolithic millirod, 10% doxorubicin, 50% PEG, 40% PLGA. (b) Membrane-encased millirod with sustained-release kinetics. PLA membrane contains 30% NaCl. (c) Membrane-encased millirod with dual-release kinetics. PLA membrane contains 25% NaCl and 10% doxorubicin. Millirods in (a) were used as the inner core for millirods in (b) and (c).

FIG. 3. SEM analysis of the morphology of NaCl-impregnated PLGA membrane. The NaCl loading percentage is 50 w/w % and the membrane thickness is 137±18 μm. (a) Surface morphology before the hydration study. (b) Surface morphology after 48 hours of hydration study. The inset in each figure shows the cross-section of the membrane. The scale bar is 10 μm in FIG. 3b inset and 100 μm in all the other images.

FIG. 4. Cumulative release (a) and rate profiles (b) of membrane-encased millirods. The structural composition for each type of millirod is listed in Table 1. The error bars in FIG. 4a were measured from triplicate samples. For clarity of presentation, the error bars were not shown in FIG. 4b.

FIG. 5. SEM analysis of the cross-section of FU-2 millirods before release study (a), 2 days (b) and 18 days (c) after release study in PBS buffer. The scale bars are 100 μm in all the images.

FIG. 6. Steady-state drug distribution as predicted by the mathematical model. We assume that rₚ=0.08 cm, rₛ=0.5 cm. The drug distribution profiles are modeled for three release rates (R_{D}=30, 60, 120 μg/cm/day).

FIG. 7. Rational design of burst dose (A_{B}) and drug release rate (R_{D}) to reach and maintain a targeted drug concentration (C_{T}) at rₛ. Three values of rₛ are evaluated at 0.3, 0.5 and 0.8 cm.

FIG. 8. Release profiles of monolithic millirods with 10, 20 and 30 w/w % loading density of 5-FU. The release studies were carried out in PBS buffer at 37° C. The error bars were measured from triplicate samples.

FIG. 9. SEM analysis of the microstructures of 10 and 30 w/w % monolithic millirods after 2 days in vitro release study. (a) 30 w/w % millirod, side surface. (b) 30 w/w % millirod, cross-section. (c) 10 w/w % millirod, side surface. (d) 10 w/w % millirod, cross-section. The scale bars are 100 μm for all the images.

FIG. 10. Cumulative release profiles of dual-release millirods. The structural composition for each type of millirod is listed in Table 2. The error bars in FIG. 10 were measured from triplicate samples. (a) Millirods with the same sustained release rate, but different burst doses. (b) Millirods with the same burst dose, but different sustained release rates.

FIG. 11. SEM analysis of the dual-release millirods (B3S2) before release (a) and seven days after release studies (b). OL: outer layer, ML: middle layer, IC: inner core. The scale bars in both images are 100 μm.

FIG. 12. Fundamental pharmacokinetic relationships for systemic administration of drugs. Dashed and dotted lines—continuous i.v. infusion; Solid line—intermittent dosing. Partially adapted from Benet, L. Z., Kroetz, D. L. and Sheiner, L. B. Pharmacokinetics. In: Hardman J G L L, Molinoff P B, Ruddon R W, ed. Goodman & Gilman's The Pharmacological Basis of Therapeutic, ed. 9th. New York: McGraw-Hill Health Professions Division, 1996; 3-27.

FIG. 13. Schematic representation of the thermoablated tumor tissue. The diameter of the millirod and the average diameter of the ablated area are denoted by rₚ and rₛ, respectively.

FIG. 14. Fluorescence imaging of doxorubicin distribution in normal and ablated rabbit livers. A, B: doxorubicin distribution in normal livers 24 and 48 hours after millirod implantation, respectively. C, D, and E: doxorubicin distribution in ablated livers 4, 24 and 48 hours after millirod implantation, respectively. The white dashed lines in C-E represent the ablated-normal tissue boundary. Due to the large distribution pattern in ablated livers, only half of the liver slice is shown in C-E. The scale bar (3 mm) in E applies to A-D as well. F: Fluorescence microscopy image (4×) of doxorubicin at the normal-ablated tissue boundary 24 hours after millirod implantation.

FIG. 15. Quantitative doxorubicin distribution profiles for different experimental conditions.

FIG. 16. Doxorubicin concentration at the ablated-normal tissue boundary 4, 24 and 48 hours after millirod implantation.

### DETAILED DESCRIPTION OF THE INVENTION

In one aspect, the present invention provides new methods and devices for dual release of therapeutic agents. In certain embodiments, the therapeutic agents are delivered locally to an area of a patient. One method of local therapy is to directly implant drug delivery devices within or immediately adjacent to the tissue or area to be treated. Exemplary applications for such devices include administering chemotherapeutic agents to the site of a tumor (e.g., alone, or in conjuction with another therapy, e.g., radiation or thermoablation), local administration of an analgesic (e.g., following an invasive procedure, such as an incision or biopsy), and antibiotic treatment (e.g., positioning a device of the present invention adjacent to a wound or lesion). The implantation may be achieved either by surgical operation or by image-guided (e.g., ultrasound, magnetic resonance imaging (MRI), or computed tomography (CT)), minimally invasive surgical procedures. One or more therapeutic agents are delivered interstitially from the device to the area for a sustained period of time.

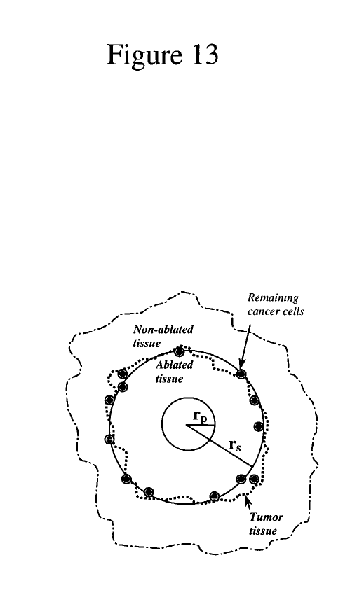

In one embodiment, the drug delivery device is fabricated in the shape of a cylindrical millirod, e.g., is suitable to be implanted directly into patient, such as, for example, adjacent to thermoablated tumor tissue, optionally under image-guided procedures (as schematically depicted by FIG. 13). These devices have several potential advantages: (1) the procedure is minimally invasive and can be carried out under local anesthesia; (2) high resolution image-guidance permits the implantation of drug delivery device within the tissue/area to be treated where it can release cancer drugs directly to the cells, such as cancer cells, in that environment; (3) sustained drug delivery can maintain the drug concentration within the therapeutic window for a prolonged period of time and improve drug efficacy; and (4) local delivery can reduce drug dosage, toxicity and other side effects that are usually associated with administration of therapeutics, especially chemotherapeutics. In certain embodiments, the device may be implanted directly inside the tumor. Of course, it is not necessary for an embodiment of the invention to present all of these advantages at once. When used in combination with RF ablation, RF ablation destroys the majority of tumor tissue and, consequently, reduces the required drug dosage for intratumoral delivery; and destruction of the tumor vasculature by RF ablation can prevent drug loss due to perfusion and, thus, improve delivery efficiency, making the combination therapy especially advantageous. The skilled artisan would readily appreciate that similar devices may be fabricated in any number of varied shapes and/or sizes, including microspheres and pellets.

Methods of implanting a drug delivery device are well known in the art, and include surgical means, injection, trocar, etc. The devices can be implanted by using an implanter, the operation of which is described in U.S. Pat. Nos. 3,921,632 and 4,451,254. Surgical procedures, such as those known in the art, may be necessary to position large implants.

Additional background information generally relating to the construction and use of drug delivery devices such as those of the present invention can be found in U.S. Pat. No. 6,331,313, which is hereby incorporated by reference for this purpose.

Definitions

For convenience, before further description of the present invention, certain terms employed in the specification, examples, and appended claims are collected here. These definitions should be read in light of the remainder of the disclosure and understood as by a person of skill in the art.

The articles “a” and “an” are used herein to refer to one or to more than one (i.e., to at least one) of the grammatical object of the article. For example, ‘an element’ means one or more than one element.

The terms “biocompatible polymer” and “biocompatibility” when used in relation to polymers are art-recognized. For example, biocompatible polymers include polymers that are neither themselves toxic to the host (e.g., an animal or human), nor degrade (if the polymer degrades) at a rate that produces monomeric or oligomeric subunits or other byproducts that are toxic or are produced at toxic concentrations in the host. In certain embodiments of the present invention, biodegradation generally involves degradation of the polymer in an organism, e.g., into its monomeric subunits, which may be known to be effectively non-toxic. Intermediate oligomeric products resulting from such degradation may have different toxicological properties, however, or biodegradation may involve oxidation or other biochemical reactions that generate molecules other than monomeric subunits of the polymer. Consequently, in certain embodiments, toxicology of a biodegradable polymer intended for in vivo use, such as implantation or injection into a patient, may be determined after one or more toxicity analyses. It is not necessary that any subject composition have a purity of 100% to be deemed biocompatible. Hence, a subject composition may comprise 99%, 98%, 97%, 96%, 95%, 90% 85%, 80%, 75% or even less of biocompatible polymers, e.g., including polymers and other materials and excipients described herein, and still be biocompatible.

To determine whether a polymer or other material is biocompatible, it may be beneficial to conduct a toxicity analysis. Such assays are well known in the art. One example of such an assay may be performed with live carcinoma cells, such as GT3TKB tumor cells, in the following manner: the sample is degraded in 1 M NaOH at 37° C. until complete degradation is observed. The solution is then neutralized with 1 M HCI. About 200 μL of various concentrations of the degraded sample products are placed in 96-well tissue culture plates and seeded with human gastric carcinoma cells (GT3TKB) at 104/well density. The degraded sample products are incubated with the GT3TKB cells for 48 hours. The results of the assay may be plotted as % relative growth vs. concentration of degraded sample in the tissue-culture well. In addition, polymers and formulations of the present invention may also be evaluated by well-known in vivo tests, such as subcutaneous implantations in rats to confirm that they do not cause significant levels of irritation or inflammation at the subcutaneous implantation sites.

The term “biodegradable” is art-recognized, and includes polymers, compositions and formulations, such as those described herein, that are intended to degrade during use. Biodegradable polymers typically differ from non-biodegradable polymers in that the former may be degraded during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to sidechain or that connects a side chain to the polymer backbone. For example, a therapeutic agent or other chemical moiety attached as a side chain to the polymer backbone may be released by biodegradation. In certain embodiments, one or the other or both generally types of biodegradation may occur during use of a polymer.

As used herein, the term “biodegradation” encompasses both general types of biodegradation. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of the implant, and the mode and location of administration. For example, the greater the molecular weight, the higher the degree of crystallinity, and/or the greater the biostability, the biodegradation of any biodegradable polymer is usually slower. The term “biodegradable” is intended to cover materials and processes also termed “bioerodible”.

In certain embodiments wherein the biodegradable polymer also has a therapeutic agent or other material associated with it, the biodegradation rate of such polymer may be characterized by a release rate of such materials. In such circumstances, the biodegradation rate may depend on not only the chemical identity and physical characteristics of the polymer, but also on the identity of material(s) incorporated therein.

In certain embodiments, polymeric formulations of the present invention biodegrade within a period that is acceptable in the desired application. In certain embodiments, such as in vivo therapy, such degradation occurs in a period usually less than about five years, one year, six months, three months, one month, fifteen days, five days, three days, or even one day on exposure to a physiological solution with a pH between 6 and 8 having a temperature of between 25 and 37° C. In other embodiments, the polymer degrades in a period of between about one hour and several weeks, depending on the desired application.

As used herein, the term “bioresorbable” means the degradative products of the material are metabolized in vivo or excreted from the body via natural pathways.

The subject compositions may contain a “drug”, “therapeutic agent,” “medicament,” or “bioactive agent,” which terms are used interchangeably herein to refer to biologically, physiologically, or pharmacologically active substances that act systemically or, preferably, locally in the human or animal body. Various forms of the medicaments or biologically active materials may be used which are capable of being released from the polymer matrix into adjacent tissues or fluids. They may be acidic, basic, or salts. They may be neutral molecules, polar molecules, or molecular complexes capable of hydrogen bonding. They may be in the form of ethers, esters, amides and the like, including prodrugs which are biologically activated when injected into the human or animal body, e.g., by cleavage of an ester or amide. An analgesic agent is also an example of a “bioactive substance.” Any additional bioactive substance in a subject composition may vary widely with the purpose for the composition. The term bioactive agent includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

Agents that may be incorporated in the subject devices include imaging and diagnostic agents (such as radioopaque agents, labeled antibodies, labeled nucleic acid probes, dyes, such as colored or fluorescent dyes, etc.) and adjuvants (radiosensitizers, transfection-enhancing agents (such as chloroquine and analogs thereof), chemotactic agents and chemoattractants, peptides that modulate cell adhesion and/or cell mobility, tissue permeabilizing agents, inhibitors of multidrug resistance and/or efflux pumps, etc.), in addition to agents that treat the patient's condition directly.

The term “drug delivery device” is an art-recognized term and refers to any medical device suitable for the application of a drug or therapeutic agent to a targeted organ or anatomic region. The term includes, without limitation, those formulations of the compositions of the present invention that release the therapeutic agent into the surrounding tissues of an anatomic area. The term further includes those devices that transport or accomplish the instillation of the compositions of the present invention towards the targeted organ or anatomic area, even if the device itself is not formulated to include the composition. As an example, a needle or a catheter through which the composition is inserted into an anatomic area or into a blood vessel or other structure related to the anatomic area is understood to be a drug delivery device. As a further example, a stent or a shunt or a catheter that has the composition included in its substance or coated on its surface is understood to be a drug delivery device.

When used with respect to a therapeutic agent or other material, the term “sustained release” is art-recognized. For example, a subject composition which releases a substance over time may exhibit sustained release characteristics, in contrast to a bolus type administration in which the entire amount of the substance is made biologically available at one time. For example, in particular embodiments, upon contact with body fluids including blood, spinal fluid, lymph or the like, the polymer matrices (formulated as provided herein and otherwise as known to one of skill in the art) may undergo gradual degradation (e.g., through hydrolysis) with concomitant release of any material incorporated therein, e.g., an therapeutic and/or biologically active agent, for a sustained or extended period (as compared to the release from a bolus). This release may result in prolonged delivery of therapeutically effective amounts of any incorporated therapeutic agent. Sustained release will vary in certain embodiments as described in greater detail below.

The term “delivery agent” is an art-recognized term, and includes molecules that facilitate the intracellular delivery of a therapeutic agent or other material. Examples of delivery agents include: sterols (e.g., cholesterol) and lipids (e.g., a cationic lipid, virosome or liposome).

“Dual release” refers to a device that releases one or more therapeutic agents at at least two different rates. In preferred embodiments, a dual-release device is a device that provides immediate release of an agent together with sustained release of that agent or of a different agent.

The term “treating” is art-recognized and includes preventing a disease, disorder or condition from occurring in an animal which may be predisposed to the disease, disorder and/or condition but has not yet been diagnosed as having it; inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain.

The phrase “pharmaceutically acceptable” is art-recognized. In certain embodiments, the term includes compositions, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” is art-recognized, and includes, for example, pharmaceutically acceptable materials, compositions or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient. In certain embodiments, a pharmaceutically acceptable carrier is non-pyrogenic. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) cocoa butter and suppository waxes; (9) oils, such as peanut oil, cottonseed oil, sunflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “pharmaceutically acceptable salts” is art-recognized, and includes relatively non-toxic, inorganic and organic acid addition salts of compositions, including without limitation, analgesic agents, therapeutic agents, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like. See, for example, J. Pharm. Sci. 66: 1-19 (1977).

A “patient,” “subject,” or “host” to be treated by the subject method may mean either a human or non-human animal, such as primates, mammals, and vertebrates.

The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic, i.e., it protects the host against developing the unwanted condition, whereas if it is administered after manifestation of the unwanted condition, the treatment is therapeutic, (i.e., it is intended to diminish, ameliorate, or stabilize the existing unwanted condition or side effects thereof).

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The phrases “systemic administration,” “administered systemically,” “peripheral administration” and “administered peripherally” are art-recognized, and include the administration of a subject composition, therapeutic or other material at a site remote from the disease being treated. Administration of an agent directly into, onto, or in the vicinity of a lesion of the disease being treated, even if the agent is subsequently distributed systemically, may be termed “local” or “topical” or “regional” administration, particularly where the agent does not reach therapeutically effective levels systemically, e.g., has a higher local concentration.

The phrase “therapeutically effective amount” is an art-recognized term. In certain embodiments, the term refers to an amount of the therapeutic agent that, when incorporated into a polymer of the present invention, produces some desired effect at a reasonable benefit/risk ratio applicable to any medical treatment. In certain embodiments, the term refers to that amount necessary or sufficient to eliminate or reduce sensations of pain for a period of time. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

The term “ED₅₀” is art-recognized. In certain embodiments, ED₅₀ means the dose of a drug that produces 50% of its maximum response or effect, or, alternatively, the dose that produces a pre-determined response in 50% of test subjects or preparations.

The term “LD₅₀” is art-recognized. In certain embodiments, LD₅₀ means the dose of a drug that is lethal in 50% of test subjects. The term “therapeutic index” is an art-recognized term that refers to the therapeutic index of a drug, defined as LD₅₀/ED₅₀.

The terms “incorporated” and “encapsulated” are art-recognized when used in reference to a therapeutic agent, or other material and a polymeric composition, such as a composition of the present invention. In certain embodiments, these terms include incorporating, formulating, or otherwise including such agent into a composition that allows for release, such as sustained release, of such agent in the desired application. The terms contemplate any manner by which a therapeutic agent or other material is incorporated into a polymer matrix, including for example: attached to a monomer of such polymer (by covalent, ionic, or other binding interaction), physical admixture, enveloping the agent in a coating layer of polymer, and having such monomer be part of the polymerization to give a polymeric formulation, distributed throughout the polymeric matrix, appended to the surface of the polymeric matrix (by covalent or other binding interactions), encapsulated inside the polymeric matrix, etc. The term “co-incorporation” or “co-encapsulation” refers to-the incorporation of a therapeutic agent or other material and at least one other therapeutic agent or other material in a subject composition.

More specifically, the physical form in which any therapeutic agent or other material is encapsulated in polymers may vary with the particular embodiment. For example, a therapeutic agent or other material may be first encapsulated in a microsphere and then combined with the polymer in such a way that at least a portion of the microsphere structure is maintained. Alternatively, a therapeutic agent or other material may be sufficiently immiscible in the polymer of the invention that it is dispersed as small droplets, rather than being dissolved, in the polymer. Any form of encapsulation or incorporation is contemplated by the present invention, in so much as the release, preferably sustained release, of any encapsulated therapeutic agent or other material determines whether the form of encapsulation is sufficiently acceptable for any particular use.

The term “biocompatible plasticizer” is art-recognized, and includes materials which are soluble or dispersible in the compositions of the present invention, which increase the flexibility of the polymer matrix, and which, in the amounts employed, are biocompatible. Suitable plasticizers are well known in the art and include those disclosed in U.S. Pat. Nos. 2,784,127 and 4,444,933. Specific plasticizers include, by way of example, acetyl tri-n-butyl citrate (c. 20 weight percent or less), acetyltrihexyl citrate (c. 20 weight percent or less), butyl benzyl phthalate, dibutylphthalate, dioctylphthalate, n-butyryl tri-n-hexyl citrate, diethylene glycol dibenzoate (c. 20 weight percent or less) and the like.

Design of the Multi-layer Drug Delivery Device

The multi-layer release drug delivery device can be fabricated in a variety of shapes and dimensions, including slabs, cylinders or tubes, films or sheets, and micro-devices (microparticles, microspheres and microcapsules). Regardless of geometry, there are at least two layers in the device. In certain embodiments, a device may comprise additional layers, e.g., between the core and the outer layers, between the two outer layers of the “three-layer” device described below, and/or around the outermost layer of a device as described below, while in other embodiments, the devices consists of, or consists essentially of, the layers described in detail below.

Structure of the Multi-layer Drug Delivery Device

A. Inner Core

The inner core of the multi-layer drug delivery device is composed of a biocompatible matrix material and a chemotherapeutic agent. Preferably, the matrix material is biodegradable and bioresorbable. Examples of biodegradeable materials are casein, albumin, calcium carbonate salts, and biodegradable polymers. Such biodegradeable polymers include, for example, polymers from the linear polyester family, such as polylactic acid, polyglycolic acid or polycaprolactone and their associated copolymers (e.g., poly(lactide-coglycolide) at all lactide to glycolide ratios and both L-lactide or D,L-lactide). Polymer types such as polyorthoester, polyanhydride, polydioxanone, and polyhydroxybutyrate may also be employed, including polysebacic acid, polyethylene glycol, and others, as well as copolymers of biodegradable polymers.

For the double-layer, dual-release delivery device it is preferred that the polymer is amorphous, i.e., it is not crystalline. It is also preferred that the polymer not generate crystalline residues upon degradation in vivo. Preferably, the in vivo lifetime of the core polymer is equal to or less than the in vivo lifetime of the outer layer polymer. For use in local drug therapy of thermoablated liver tumors, the in vivo lifetime of the core polymer is greater than 180 days. Other tumors that can be treated with devices as described herein include breast, ovarian, lung, colon, bone, skin, prostate, bladder, and other suitable cancers. In certain embodiments, the present devices can be used as polymeric spacers between brachytherapy seeds, e.g., to provide chemotherapy as an adjunct to radiotherapy. So that the drug concentration inside the device can remain saturated, the inner core may be designed to have faster release kinetics so that the final release rate will be controlled by the permeability of the outer layer, rather than being influenced by the rate of dissolution of the core.

In certain embodiments, the inner core is a monolithic device formed by a biocompatible polymer mixed with the therapeutic agent. In other embodiments the core is formed totally from soluble materials, such as PEG mixed with drug or even the pure drug itself. Preferably, the inner core, by itself and without an outer layer, is designed to release at least 90% of the drug within 24 hours after placement into a thermoablated tissue.

The inner core may contain one or more adjuvant substances, such as fillers, thickening agents or the like. In other embodiments, materials that serve as adjuvants may be associated with a polymer matrix. Such additional materials may affect the characteristics of the polymer matrix that results.

For example, fillers, such as bovine serum albumin (BSA) or mouse serum albumin (MSA), may be associated with the polymer matrix. In certain embodiments, the amount of filler may range from about 0.1 to about 50% or more by weight of the polymer matrix, or about 2.5, 5, 10, 25, or 40 percent. Incorporation of such fillers may affect the biodegradation of the polymeric material and/or the sustained release rate of any encapsulated substance. Other fillers known to those of skill in the art, such as carbohydrates, sugars, starches, saccharides, celluloses and polysaccharides, including mannitose and sucrose, may be used in certain embodiments in the present invention.

In certain embodiments, a subject composition includes an excipient. A particular excipient may be selected based on its melting point, solubility in a selected solvent (e.g., a solvent that dissolves the polymer and/or the therapeutic agent), and the resulting characteristics of the composition. Excipients may comprise a few percent, about 5%, 10%, 15%, 20%, 25%, 30%, 40%, 50%, or higher percentage of the subject compositions.

Buffers, acids and bases may be incorporated in the subject compositions to adjust their pH. Agents to increase the diffusion distance of agents released from the polymer matrix may also be included. Hyaluronidase may be included as a permeability-enhancing agent.

Outer Layer of the Double-layer Delivery Device

The outer layer of the double-layer device is formed from a biocompatible matrix material mixed with both a bioactive agent, such as an anticancer drug (5-FU, doxorubicin, etc.), and, preferably, other water-soluble components. The matrix material can be a biodegradable polymer, such as PLGA, PLA, or a non-degradable material, such as ethylene-vinyl acetate copolymer (EVAc) or polyurethane. Suitable polymers include homopolymers, copolymers, straight, branched-chain, or cross-linked derivatives. Some exemplary polymers include: polycarbamates or polyureas, cross-linked poly(vinyl acetate) and the like, ethylene-vinyl ester copolymers having an ester content of 4 to 80% such as ethylene-vinyl acetate (EVA) copolymer, ethylene-vinyl hexanoate copolymer, ethylene-vinyl propionate copolymer, ethylene-vinyl butyrate copolymer, ethylene-vinyl pentanoate copolymer, ethylene-vinyl trimethyl acetate copolymer, ethylene-vinyl diethyl acetate copolymer, ethylene-vinyl 3-methyl butanoate copolymer, ethylene-vinyl 3-3-dimethyl butanoate copolymer, and ethylene-vinyl benzoate copolymer, or a mixture thereof. The molecular weights of polymers employed may vary, e.g., between 10 kD to 500 kD, depending on the time frame desired for sustained release.

Additional examples include polymers such as: poly(methyl methacrylate), poly(butyl methacrylate), plasticized poly(vinyl chloride), plasticized poly(amides), plasticized nylon, plasticized soft nylon, plasticized poly(ethylene terephthalate), natural rubber, silicone, poly(isoprene), poly(isobutylene), poly(butadiene), poly(ethylene), poly(tetrafluoroethylene), poly(vinylidene chloride), poly(acrylonitrile), cross-linked poly(vinylpyrrolidone), chlorinated poly(ethylene), poly(trifluorochloroethylene), poly(ethylene chlorotrifluoroethylene), poly(tetrafluoroethylene), poly(ethylene tetrafluoroethylene), poly(4,4′-isopropylidene diphenylene carbonate), polyurethane, poly(perfluoroalkoxy), poly(vinylidene fluoride), vinylidene chloride-acrylonitrile copolymer, vinyl chloride-diethyl fumarate copolymer, silicone, silicone rubbers (of medical grade, such as Silastic® Medical Grade ETR Elastomer Q7-4750 or Dow Coming® MDX 4-4210 Medical Grade Elastomer); and cross-linked copolymers of polydimethylsiloxane silicone polymers.

Some further examples of polymers include: poly(dimethylsiloxanes), ethylene-propylene rubber, silicone-carbonate copolymers, vinylidene chloride-vinyl chloride copolymer, vinyl chloride-acrylonitrile copolymer, vinylidene chloride-acrylonitrile copolymer, poly(olefins), poly(vinyl-olefins), poly(styrene), poly(halo-olefins), poly(vinyls) such as polyvinyl acetate, cross-linked polyvinyl alcohol, cross-linked polyvinyl butyrate, ethylene ethylacrylate copolymer, polyethyl hexylacrylate, polyvinyl chloride, polyvinyl acetals, plasticized ethylene vinylacetate copolymer, polyvinyl alcohol, polyvinyl acetate, ethylene vinyl chloride copolymer, polyvinyl esters, polyvinyl butyrate, polyvinylformal, poly(acrylate), poly(methacrylate), poly(oxides), poly(esters), poly(amides), and poly(carbonates), or a mixture thereof.

In some aspects, the devices may be biodegradable wherein the outer layer degrades after the drug has been released for the desired duration. The biodegradable polymeric compositions may comprise organic esters or ethers, which when degraded result in physiologically acceptable degradation products, including the monomers. Anhydrides, amides, orthoesters, or the like, by themselves or in combination with other monomers, may find use. The polymers may be addition or condensation polymers, cross-linked or non-cross-linked. For the most part, besides carbon and hydrogen, the polymers will include oxygen and nitrogen, particularly oxygen. The oxygen may be present as oxy, e.g., hydroxy, ether, carbonyl, e.g., carboxylic acid ester, and the like. The nitrogen may be present as amide, cyano, or amino. In some aspects, the polymer is polytetrafluoroethylene, (commercially known as Teflon®), ethyl vinyl alcohol or ethylene vinyl acetate.

Some examples of biodegradable polymers useful in the present invention include: hydroxyaliphatic carboxylic acids, either homo- or copolymers, such as polylactic acid, polyglycolic acid, polylactic glycolic acid; polysaccharides such as cellulose or cellulose derivatives such as ethyl cellulose, crosslinked or uncrosslinked sodium carboxymethyl cellulose, sodium carboxymethylcellulose starch, cellulose ethers, cellulose esters such as cellulose acetate, cellulose acetate phthalate, hydroxypropylmethyl cellulose phthalate and calcium alginate, polypropylene, polybutyrates, polycarbonate, acrylate polymers such as polymethacrylates, polyanhydrides, polyvalerates, polycaprolactones such as poly-ε-caprolactone, polydimethylsiloxane, polyamides, polyvinylpyrrolidone, polyvinylalcohol phthalate, waxes such as paraffin wax and white beeswax, natural oils, shellac, zein, or a mixture thereof.

The water-soluble (pore-forming) component can be salt particles (NaCl, KCl, etc.), soluble polymers (PEG, Pluronic, etc.) or other soluble compounds such as glucose. In preferred embodiments, the water-soluble component is substantially evenly distributed throughout the layer, i.e., the water-soluble component is randomly dispersed through the layer rather than being located in a particular region of the layer, e.g., such that the membrane becomes porous over substantially the entire surface of the device, and the drug contained in the core diffuses through the membrane in substantially all directions. Various methods can be utilized to form an outer layer over the inner core—a process that will result in the double-layer structure. These include: (1) Preparation of film by solvent casting or heat-compression molding followed by ‘wrapping’ of the film around the inner core. 2) Dip-coating of the inner core in a concentrated solution of the outer layer materials in a low boiling point organic solvent followed by air and vacuum drying of the device. (3) Embedding of the inner core into a solid powder mixture of the outer layer materials followed by application of compression or heat-compression molding. The loading percentage of water-soluble material and its morphology (size and shape) controls the diffusivity of drug through the outer semi-permeable membrane once the pore-forming material is dissolved. Furthermore, if the membrane material is biodegradable, its degradation time will limit the duration of release. The invention contemplates that a plurality of pores, rather than a single pore or a few specially placed pores, will be present after dissolution of the pore-forming material.

With respect to the pore-forming agent, any biocompatible water-soluble material may be used as the pore-forming agent. They may be capable of dissolving, diffusing or dispersing out of the formed polymer system whereupon pores and microporous channels are generated in the system. The amount of pore-forming agent (and size of dispersed particles of such pore-forming agent, if appropriate) within the composition should affect the size and number of the pores in the polymer system, and thus affect the eventual rate of release of active agent from the inner core.

Pore-forming agents include any pharmaceutically acceptable organic or inorganic substance that is substantially miscible in water and body fluids and will dissipate from the forming and formed matrix into aqueous medium or body fluids or water-immiscible substances that rapidly degrade to water-soluble substances. Suitable pore-forming agents include, for example, sugars such as sucrose and dextrose, salts such as sodium chloride and sodium carbonate, and polymers such as hydroxylpropylcellulose, carboxymethylcellulose, polyethylene glycol, and PVP. The size and extent of the pores may be varied over a wide range by changing the molecular weight, particle size, and loading of pore-forming agent incorporated into the polymer system.

Outer Layers of Triple-Layer Delivery Device

A triple-layer delivery device adds a third layer to the two discussed above, although in these embodiments, the middle layer need not comprise a bioactive agent. The third layer of the triple-layer delivery device comprises a hydrophilic polymer and a therapeutic agent, such as an anticancer drug. Hydrophilic polymers (such as PEG, gelatin, or dextran) provide a cohesive surface coating before the implantation and a fast dissolution to introduce the burst dose after implantation. The techniques for the formation of water-soluble polymer coatings on a matrix are routinely used in pharmaceutical and other industries. Methods such as dip-dry or spray coating can be applied to achieve this goal. (Tracton, A. (2001) Coatings technology handbook; Carstensen, J. T. (2001) New York: Marcel Dekker; Bauer, K. H. (1998) Stuttgart: Medpharm Scientific Publishers; Boca Raton:CRC Press.) Alternatively, the third layer comprises a chemotherapeutic agent and a water-soluble inorganic material (or any pore-forming agent, as discussed above), such as for example, hydroxyapatite. The three-layer device may be more useful to deliver two or more therapeutic agents from a millirod device. The release kinetics can be dual release, but can also permit other possibilities, such as sequential release of one drug before another, etc. Plasticizers and stabilizing agents known in the art may be incorporated in membranes of the present devices. In certain embodiments, additives such as plasticizers and stabilizing agents are selected for their biocompatibility.

Bioactive Agents

As described above, device contains one or more agents effective in obtaining a desired local or systemic physiological or pharmacological effect. The following classes of agents could be incorporated into the devices of the present invention: anesthetics and analgesic agents such as lidocaine and related compounds and benzodiazepam and related compounds; anti-cancer agents such as 5-fluorouracil, adriamycin and related compounds; antiinflammatory agents such as 6-mannose phosphate; anti-fungal agents such as fluconazole and related compounds; anti-viral agents such as trisodium phosphomonoformate, trifluorothymidine, acyclovir, ganciclovir, DDI and AZT; cell transport/mobility impending agents such as colchicine, vincristine, cytochalasin B and related compounds; antiglaucoma drugs such as beta-blockers: timolol, betaxolol, atenalol, etc.; immunological response modifiers such as muramyl dipeptide and related compounds; peptides and proteins such as cyclosporin, insulin, growth hormones, insulin related growth factor, heat shock proteins and related compounds; and steroidal compounds such as dexamethasone, prednisolone and related compounds, including low-solubility steroids such as fluocinolone acetonide and related compounds.

In addition to the above agents, other agents include neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antibacterials such as sulfonamides, sulfacetamide, sulfamethizole and sulfisoxazole; antivirals, including idoxuridine; and other antibacterial agents such as nitrofurazone and sodium propionate; antiallergenics such as antazoline, methapyriline, chlorpheniramine, pyrilamine and prophenpyridamine; angiogenesis inhibitors, such as angiostatin and endostatin; permeability enhancers such as hyaluronidase; anti-inflammatories such as hydrocortisone, hydrocortisone acetate, dexamethasone 21-phosphate, fluocinolone, medrysone, methylprednisolone, prednisolone 21-phosphate, prednisolone acetate, fluoromethalone, betamethasone and triminolone; decongestants such as phenylephrine, naphazoline, and tetrahydrazoline; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as those described in Design of Prodrugs, edited by Hans Bundgaard, Elsevier Scientific Publishing Co., Amsterdam, 1985. Once again, reference may be made to any standard pharmaceutical textbook such as Remington's Pharmaceutical Sciences for the identity of other agents.

Anticlotting agents such as heparin, antifibrinogen, fibrinolysin, anti clotting activase, etc., can also be delivered. Antidiabetic agents that may be delivered using the present devices include acetohexamide, chlorpropamide, glipizide, glyburide, tolazamide, tolbutamide, insulin, aldose reductase inhibitors, etc.

Hormones, peptides, nucleic acids, saccharides, lipids, glycolipids, glycoproteins, and other macromolecules can be delivered using the present devices. Examples include: endocrine hormones such as pituitary, insulin, insulin-related growth factor, thyroid, growth hormones; heat shock proteins; immunological response modifiers such as muramyl dipeptide, cyclosporins, interferons (including α, β, and γ interferons), interleukin-2, cytokines, FK506 (an epoxy-pyrido-oxaazacyclotricosine-tetrone, also known as Tacrolimus), tumor necrosis factor, pentostatin, thymopentin, transforming factor beta₂, erythropoetin; antineogenesis proteins (e.g., anit VEGF, Interferons), among others and anticlotting agents including anticlotting activase. Further examples of macromolecules that can be delivered include monoclonal antibodies, brain nerve growth factor (BNGF), ciliary nerve growth factor (CNGF), vascular endothelial growth factor (VEGF), and monoclonal antibodies directed against such growth factors. Additional examples of immunomodulators include tumor necrosis factor inhibitors such as thalidomide.

In embodiments relating to the treatment of cancer, any of a variety of chemotherapeutic agents can be used in the multi-layer drug delivery device for sustained-release delivery. The classes of applicable chemotherapeutic agents include DNA alkylating agents (e.g., BCNU, cisplatin, carboplatin), antimetabolites (e.g., 5-FU, methotrexate), antibiotics (e.g., doxorubicin, bleomycin), vinca alkaloids, and hormones (e.g., prednisone, leuprolide). Some examples of anti-cancer agents include 5-fluorouracil, adriamycin, asparaginase, azacitidine, azathioprine, bleomycin, busulfan, carboplatin, carmustine, chlorambucil, cisplatin, cyclophosphamide, cyclosporine, cytarabine, dacarbazine, dactinomycin, daunorubicin, doxorubicin, estramustine, etoposide, etretinate, filgrastin, floxuridine, fludarabine, fluorouracil, fluoxymesterone, flutamide, goserelin, hydroxyurea, ifosfamide, leuprolide, levamisole, lomustine, nitrogen mustard, melphalan, mercaptopurine, methotrexate, mitomycin, mitotane, pentostatin, pipobroman, plicamycin, procarbazine, sargramostin, streptozocin, tamoxifen, taxol, teniposide, thioguanine, uracil mustard, vinblastine, vincristine and vindesine. Dosages may be optimized depending on the size of the tumor, the release time, drug potency, and implant size.

Certain additional agents may be included in the core and/or the membrane(s) of a device of the present invention. Binders are adhesive materials that may be incorporated in polymeric formulations to bind and maintain matrix integrity. Binders may be added as dry powder or as solution. Sugars and natural and synthetic polymers may act as binders.

Materials added specifically as binders are generally included in the range of about 0.5%-15% w/w of the matrix formulation. Certain materials, such as microcrystalline cellulose, also have additional binding properties.

The present compositions may additionally contain one or more optional additives such as fibrous reinforcement, colorants, perfumes, rubber modifiers, modifying agents, etc. In practice, each of these optional additives should be compatible with the resulting polymer and its intended use. Examples of suitable fibrous reinforcement include PGA microfibrils, collagen microfibrils, cellulosic microfibrils, and olefinic microfibrils. The amount of each of these optional additives employed in the composition is an amount necessary to achieve the desired effect.

### COMPARATIVE EXAMPLES

Development of polymer millirods with “burst” release of doxorubicin. These devices are prepared as a monolithic system using a compression-heat-molding procedure that has been developed. (Qian, F., Szymanski, A. and Gao, J. (2001) J. Biomed. Mater. Res. 55, 512-522.) Briefly, doxorubicin solution (2 mg/mL, also containing 0.9% NaCl) from Bedford Laboratories is first dialyzed in distilled water to remove the NaCl. The desalted solution is lyophilized to obtain the purified solid particles of doxorubicin. The drug particles are then mixed with PLGA (G:A=1:1, Mn=50 kD) microspheres (diameter: 5 μm) and poly(ethylene glycol) (PEG) particles. The well-mixed particles are placed in a Teflon tube and molded into a polymer millirod at a compression pressure of 4.6×10⁶ Pa and a fabrication temperature at 90° C. for 2 hours. Previous studies have shown that this fabrication procedure produced polymer millirods with reproducible release kinetics and adequate mechanical strength for implantation. In addition, the structure of doxorubicin remained intact after fabrication as shown by nuclear magnetic resonance spectroscopy (NMR).

As shown below in Example 1, incorporation of 50% PEG in the polymer matrix permits the “burst” release of doxorubicin from the polymer millirods. It should be noted that the loading of PEG is important to achieve “burst” release kinetics in these monolithic systems, especially when the loading density of doxorubicin may be low. Drug diffusion is the main mechanism to achieve a burst release of doxorubicin within 24 hours since the half-weight degradation time for PLGA polymer is approximately 4 weeks (Sawhney, A. S. and Hubbell, J. A. (1990) J. Biomed. Mater. Res. 24, 1397-1411).

When the doxorubicin loading is low, it is desirable to raise the PEG loading above the percolation threshold in the PLGA matrix to form interconnecting pores and channels to allow fast release of drugs. These millirods are used to evaluate the effect of loading density of PEG on the release kinetics of doxorubicin from polymer millirods. This type of release kinetics can be described by Higuchi's model (t^{½} release) at initial phase and by an exponential model (e^{-kt} release) at later phase. (Baker, R. (1987) New York: Wiley.)

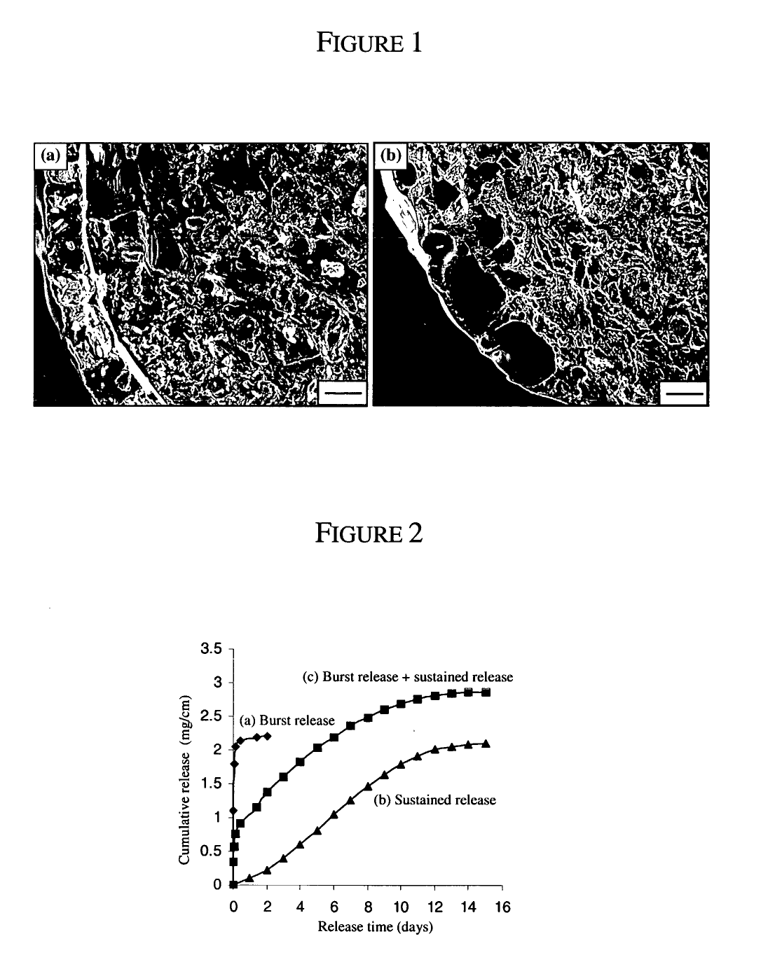

Fabrication of multi-layer polymer millirods with “sustained” release of doxorubicin. We have established a novel design of a membrane-encased polymer millirod for the sustained release of doxorubicin. In this device, a polymer millirod with burst release is chosen as an inner core, and it is encapsulated by a polymer membrane to form a membrane-encased millirod. The polymer membrane serves as a barrier material to provide a sustained release of doxorubicin. Here we choose PLA (Mn=88 kD) over PLGA as the membrane material because PLA degrades much slower than PLGA. For example, the half-weight degradation time of PLA is 155 days compared to 30 days for PLGA. (Sawhney, A. S. and Hubbell, J. A. (1990) J. Biomed. Mater. Res. 24, 1397-1411.) As shown in Example 1 below, the use of PLA film achieved sustained release over two weeks, a time period that is suitable for the proposed application (FIG. 2).

The polymer membrane is fabricated by a solvent evaporation procedure. Typically, PLA polymer is dissolved at 200 mg/mL in methylene chloride. Sieved NaCl particles of a specific size (e.g., 38-90 μm) are added in the polymer solution. The suspension is mixed and poured into a Teflon petri dish (diameter: 10 cm). The solvent is allowed to evaporate overnight in the fume hood and the film is further dried in vacuo for two days. After drying, the polymer film is removed from petri dish and wrapped around the inner millirod. The film ends are sealed by heat molding to yield the final membrane-encased millirods.

The prototype millirods are useful for studying the mechanism of sustained release in these systems. For example, the millirods can provide a coherent mechanism to explain the sustained and relatively constant rate of release from these millirods. The membrane-encased millirods are removed from buffer at different time points and the changes in weight, volume (swelling) and morphology at the surface, cross-section and interior (by SEM) of the millirods are determined. In addition, the millirods can be used to evaluate the processes of salt leaching in PLA membrane and water diffusion into the device. The millirods can be used to assess the effect of drug loading and release kinetics of the inner millirod on the overall release rate and time. Results from this study can also shed light on the mechanism of sustained release. Finally, the millirods can be used to evaluate the following parameters—film thickness, density of NaCl particle in the membrane (potentially affecting porosity) and the size of NaCl particles (tortuosity)—to control the membrane diffusivity and the subsequent release rate of doxorubicin from polymer millirods. These studies will provide fundamental knowledge for the device design and establish useful parameters for the accurate control of drug release rate and time duration. Finally, the double-layer millirods can be used systematically to deliver chemotherapeutic agents to patients.

Fabrication of polymer millirods with “dual” release of doxorubicin. The development of polymer millirods with dual-release kinetics is closely associated with the development for the sustained device. In fact, the techniques involved in the sustained-release system can be directly utilized for the design and development of dual release millirods.

As shown in Example 1 below, we achieved the dual release of doxorubicin using the same two-component design as the membrane-encased polymer millirod. By impregnating both drug and NaCl particles in the PLA membrane, we achieved burst release of doxorubicin from PLA membrane in the first 10 hours and a sustained release from inner rod for another 2 weeks (FIG. 2). In this approach, the burst dose is controlled by adjusting the amount of doxorubicin impregnated in the PLA membrane, and the sustained-release rate (R_{D}) is controlled by the porous membrane left by the leaching of NaCl and doxorubicin particles.

Millirod Characterization

Scanning electron microscopy (SEM). SEM (model 840, JEOL) was used to examine the surface and cross sections of the polymer millirods. SEM can provide information relating to homogeneity of NaCl crystals and the resulting pores in the PLA film, thickness of PLA films and coating homogeneity, change of membrane structure (porosity, tortuosity) over time during release, etc. Before SEM analysis, samples were mounted on the aluminum stub by double-sided tape and sputter coated with Pd (10 nm thickness). Typically, SEM analysis was carried out at an accelerating voltage of 20 kV.

Measurement of drug release profiles in vitro. The drug release is measured in phosphate-buffered saline (PBS, pH 7.4) at 37° C. by a UV-Vis spectrophotometer (Lambda 20 model, Perkin-Elmer Corp.). In a typical procedure, segments of millirods (˜10 mm in length) are weighed prior to the drug release. The rods are placed in 20 mL glass vials and submerged in 5 mL PBS buffer. The vials are placed in an orbital shaker at 37° C., and 5 mL sample solution is removed periodically for UV measurement. After sample removal, the sample vial is refilled with 5 mL fresh buffer. The Beer-Lambert law is used to calculate the agent concentration at its maximum absorption wavelength (λₘₐₓ=480 nm). The cumulative mass of the released agent is calculated by adding the individual sample mass after each removal. The release profile is obtained by plotting the amount of released agent as a function of time.

Determination of the structural integrity of released doxorubicin. Released doxorubicin in PBS is desalted by gel filtration column chromatography (G-10 column, Pharmacia). Samples are lyophilized and characterized by ¹H and ¹³C nuclear magnetic resonance (NMR) and mass spectrometry to examine their structural integrity. In NMR analysis, lyophilized agents are dissolved in deuterated methanol and spectrum will be obtained on a 300 MHz ¹H NMR instrument (Bruker). The chemical shifts of the released agents are compared to the pure compound to detect possible structural decomposition.

Exemplification

The invention now being generally described, it will be more readily understood by reference to the following examples, which are included merely for purposes of illustration of certain aspects and embodiments of the present invention, and are not intended to limit the invention.

### EXAMPLE 1

Doxorubicin-containing polymer millirods with monolithic or membrane-encased structure were fabricated. We chose poly(D,L-lactic-co-glycolic acid) (PLGA) and poly(D,L-lactic acid) (PLA) as the polymer matrix. The monolithic millirods were fabricated by a heat-compression molding procedure (Feng, Q., Szymanski, A. and Gao, J. (2001) J. Biomed. Mater. Res. 55, 512-522), where polymer microspheres and doxorubicin particles were physically mixed and molded at a temperature (90° C.) higher than the glass transition temperature of PLGA (T_{g}=45° C.) under compression pressure (4.6×10⁶ Pa). Membrane-encased millirods were fabricated by wrapping and annealing a PLA film (thickness 150 μm) over an inner monolithic millirod. The outer PLA membrane contained either solely NaCl particles or both NaCl and doxorubicin particles. The detailed fabrication procedure is described below.

FIG. 1 shows the cross-section of an exemplary membrane-encased millirod before and after the salt leaching experiments. In this example, the outer PLA membrane contained 20% NaCl particles (size 90-150 μm). It is obvious that after salt leaching, channels and pores were formed across the outer membrane. This membrane became a semi-permeable barrier to control the release rate of doxorubicin from the inner millirod.

FIG. 2 shows the release profiles of three types of polymer millirods with different release kinetics. The monolithic millirod with the composition of 10% doxorubicin, 50% PEG and 40% PLGA gave rise to a burst release profile, where all the doxorubicin molecules were released in about 10 hours (2a). The same monolithic millirod encased in a PLA membrane with 30% NaCl particles led to a sustained release of doxorubicin over 2 weeks (2b). The average release rate was approximately 150 μm/day/cm length of millirod in the first two weeks. The membrane-encased millirod with an outer membrane containing NaCl (25%) and doxorubicin (10%) particles resulted in initial burst release followed by sustained release for about 2 weeks (2c). The burst dose was 0.95 mg/cm and the release rate was approximately 120 μm/day/cm. The thickness of the outer membrane in both cases was 150 μm. The release rate of the membrane-encased millirods can be controlled by adjusting the membrane thickness and loading percentage and/or particle size of NaCl in the membrane. The amount of drug released in the bust phase can also be controlled by varying the drug loading percentage in the outer membrane.

These results demonstrate the feasibility of biomaterial technology for the fabrication of polymer millirods with modulated release kinetics.

In another aspect, the present invention provides a mathematical model and method for optimizing the design of the dual release device for local chemotherapy.

Mathematical Model

The present invention also relates to mathematical models for describing the dynamics of drug distribution in thermoablated and surrounding tissues. These models take into account the different molecular transport processes in drug diffusion and drug uptake in thermoablated and non-ablated tissues. The models are used to obtain the design criteria for the initial burst dose and sustained release rate to achieve a predetermined concentration at the thermoablation boundary. Optimization of the device involves integration between the mathematical model and testing of prototype devices. The models provide a rational approach to design the drug delivery systems, and experimental results provide feedback information to refine the models for further optimal design.

Mass transport model and boundary conditions. The present model starts with a simple mass transport model to demonstrate the concept of rational design of polymer millirods with dual-release kinetics. In this model, a degradable polymer millirod is placed in an interior region of the tissue that has been thermally ablated (FIG. 13) and doxorubicin enters the liver from the rod. Although the rod radius rₚ(t) changes with time, its time constant is expected to be much larger than that for the drug transport within the tissue; consequently, the rod radius is considered as a quasi-steady constant rₚ in this model. We define the radius of the ablated tissue as rₛ from the center of the polymer rod. Within the ablated tissue where no viable cells or blood vessels exist (as supported by histology analysis below), only diffusion occurs (we assume there is no convection flow in this starting model). Since the length of the millirod (10 mm) is much greater than the diameter (1.6 mm), we assume that the dominant changes can be described using cylindrical symmetry. Consequently, the drug concentration changes in the ablated region according to:

$$\frac{\partial {C}_{a}}{\partial t} = \frac{{D}_{a}}{r} \frac{\partial [ r \partial {C}_{a} / \partial r ]}{\partial r} , {r}_{P} < r < {r}_{s}$$

where Cₐ and Dₐ are the doxorubicin concentration and diffusivity within the ablated tissue, respectively. Initially, no drug is present in the tissue:

Cₐ(r, 0)=0

At the interface between the polymer rod surface and ablated region (r=rₚ), the rate of drug release equals the rate of diffusion:

$$r = {r}_{P} : {R}_{D} ( t ) = - D \frac{\partial {C}_{a}}{\partial r}$$

where R_{D}(t) is the rate of release, which is a constant for zero-order release millirods.

At the interface between the ablated region and the surrounding viable tissue, we consider both the concentration and flux of drug are continuous:

$$r = {r}_{s} : {C}_{a} = {C}_{n} and {D}_{a} \frac{\partial {C}_{a}}{\partial r} = {D}_{n} \frac{\partial {C}_{n}}{\partial r}$$

where Cₙ and Dₙ are the doxorubicin concentration and diffusivity in the non-ablated tissue, respectively.

When the drug diffuses into the surrounding viable tissue, the drug will be taken up by the cells or lost by perfusion, so the governing equation becomes:

$$\frac{\partial {C}_{n}}{\partial t} = \frac{{D}_{n}}{r} \frac{\partial [ r \partial {C}_{n} / \partial r ]}{\partial r} - K \cdot {C}_{n} , {r}_{s} \leq r < ∞$$

where K is the combined coefficient of cell uptake and drug perfusion. With significant drug uptake processes, we expect that the drug concentration is negligible far enough from the rod. In other words, the tissue can be regarded as infinite with respect to the distant boundary condition: Cₙ(∞, t)=0.

Steady-state solutions. For zero-order release millirods or millirods having initial burst followed by zero-order release, R_{D} will be either always constant, or constant after the “burst” time, respectively. In these cases, after sufficient time, this mathematic model reaches a steady state where drug concentration is only a function of distance but not time. There exists an analytical solution for the steady state:

$$\begin{matrix}{C}_{a} = \frac{{R}_{D} {r}_{p}}{{D}_{a}} ln ( \frac{{r}_{s}}{r} ) + {C}_{s} , {r}_{P} < r < {r}_{s} \\ {C}_{n} = {C}_{s} \cdot \frac{{k}_{0} ( {β}_{r} )}{{k}_{0} ( {β}_{r} )} , {r}_{s} \leq r < ∞\end{matrix}$$

where k₀(βᵣ) is the zero-order modified Bessel functions of the second kind. β is the following constant:

$$β = \sqrt{\frac{K}{{D}_{n}}}$$

Cₛ is the steady state concentration at the boundary of ablated and normal liver tissue (r=rₛ):

$${C}_{s} = RD [ \frac{{r}_{p} \cdot {k}_{0} ( β {r}_{s} )}{β \cdot {r}_{s} \cdot {D}_{n} \cdot {k}_{1} ( β {r}_{s} )} ]$$

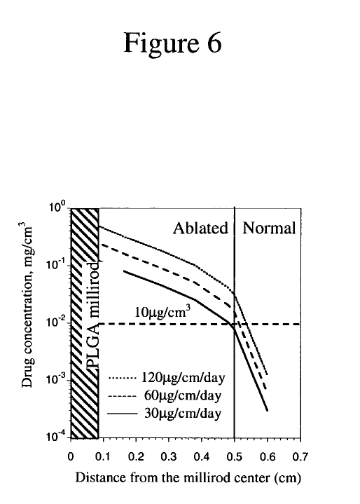

FIG. 6 shows the steady-state drug distribution profiles corresponding to millirods with different release rates. For convenience of millirod design, the unit of R_{D} is presented as the μg of doxorubicin released per centimeter length of millirod per day (μg/cm/day). In this simulation, we used parameters (Dₙ=4.3×10⁻⁷ cm²S⁻¹, K=4.4×10⁻⁴ s⁻¹) from the literature as reported by Dr. Saltzman's group (Fung, L. K., Shin, M., Tyler, B., Brem, H. and Saltzman, W. M. (1996) Pharm Res 13(5), 671-82; Strasser, J. F., Fung, L. K., Eller, S., Grossman, S. A. and Saltzman, W. M. (1995) J Pharmacol Exp Ther 275(3), 1647-55) and also assumed Dₐ=Dₙ.

Rational design of release rates and burst dose. At steady state, the value of Cₛ is proportional to R_{D} from polymer millirods. Therefore, to achieve the boundary drug concentration at a targeted value, C_{T}, the drug release rate should be controlled at:

$$RD = {C}_{T} \cdot [ \frac{β \cdot {r}_{s} \cdot {D}_{n} \cdot {k}_{1} ( β {r}_{s} )}{{r}_{p} \cdot {k}_{0} ( β {r}_{s} )} ]$$

To maintain this concentration distribution profile for a predetermined time period (t_{T}), the maintenance dosage (A_{M}) can be calculated as:

A_{M}=R_{D}·t_{T}

Before reaching the steady state, however, drug has to be released from the millirod to create the steady-state drug distribution profile. Here we define the amount of drug contained within a 1 cm thick liver tissue at steady-state distribution as the burst dose, A_{B}. The value of A_{B} can be calculated from the integration of steady state distribution profile (FIG. 6) in a cylindrical coordinate:

$$\begin{matrix}AB = {{\int }^{{r}_{s}}}_{{r}_{p}} 2 π r \cdot {C}_{a} ( r ) ⅆ r + {{\int }_{{r}_{s}}}^{∞} 2 π r \cdot {C}_{n} ( r ) ⅆ r \\ = \frac{π \cdot RD \cdot {r}_{p}}{2 {D}_{a}} [ 2 {r}_{p}^{2} ln ( \frac{{r}_{p}}{{r}_{s}} ) + {r}_{s}^{2} - {r}_{p}^{2} ] + π {C}_{T} ( {r}_{s}^{2} - {r}_{p}^{2} ) + \frac{ 2 π \cdot RD \cdot {r}_{p}}{{D}_{n}}\end{matrix}$$

The total drug dosage, A, within the millirod is therefore the sum of the burst dose and the maintenance dose:

A=A_{B}+A_{M}

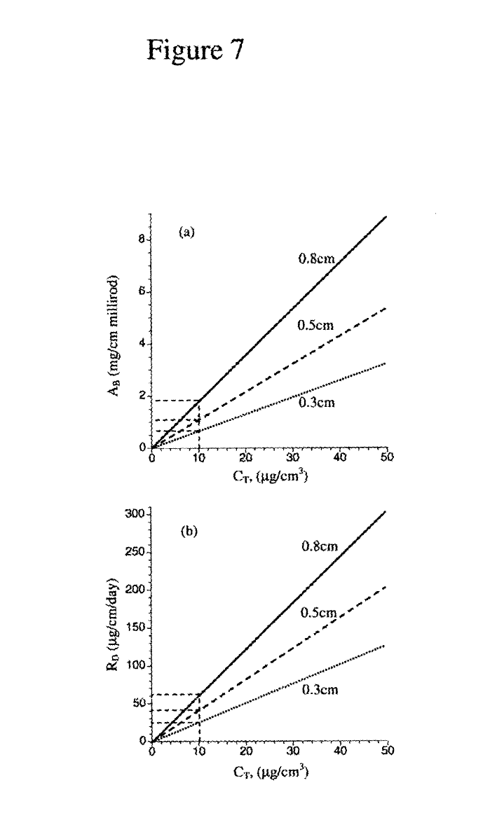

FIG. 7 illustrates the concept for the rational design of polymer millirods. Here the values of A_{B} and R_{D} are plotted as a function of C_{T} at the thermoablation boundary for three ablation sizes. Once the target concentration and ablation size are defined, the values of A_{B} and R_{D} can be identified as the design criteria for the release properties for the polymer millirods. For example, to achieve a 10 μg/cm³ concentration at the boundary tissue for 0.5 cm ablation size, the values of A_{B} and R_{D} are 1.1 mg/cm millirod and 40 μg/cm/day, respectively. These estimated values illustrate a clear advantage for introducing the burst dose in the millirod design. Without it, it will take a zero-order release device approximately 27 days to reach the drug distribution pattern in the steady-state. With the burst dose, it can take as little as 24 hours to reach the therapeutic concentration.

Parameter determination or estimation. From experimental data, it is possible measure concentrations at various distances from the polymer millirod by fluorescence imaging, and thus empirically determine the actual values of parameters for the equations above for a given biological environment. In some instances, experimentally determined values may be found in the scientific literature. Estimates of model parameters can be made using data from animal experiments with polymer millirods having dual-release kinetics. By least-square fitting of model output to the experimental data, one can obtain estimates of the model parameters: Dₐ, Dₙ and K.

Optimal parameter estimates may alternatively be obtained by minimizing the least-squares objective function computed from the residual vector of the model output and experimental data. For this purpose, one can use an adaptive, nonlinear, least-squares optimization algorithm, NL2SOL (Dennis, J. E., Gay, D. M. and Welsch, R. E (1981) ACM Trans. Math. Softw. 7, 369-383) which is available from NETLIB (http://www.netlib.org/liblist.html in toms/573). Once parameter estimation is completed, the model may be used to provide further design of polymer millirods.

### EXAMPLE 2

Liver Tumors and Doxorubicin

Dynamics of Doxorubicin Distribution in Thermoablated Rabbit Livers

In accordance with the present invention, we developed a fluorescence imaging method to analyze doxorubicin distribution in rabbit liver tissues. First, we determined the calibration curve to correlate fluorescence intensity with doxorubicin concentration in liver tissues. Second, we estimated the sensitivity limit (0.6 μg/g of liver tissue) for doxorubicin detection by this method. This value is well below the cytotoxic concentrations of doxorubicin to VX-2 cells (˜6.4 μg/g). (Ridge, J. A., Collin, C., Bading, J. R., Hancock, C., Conti, P. S., Daly, J. M. and Raaf, J. H. (1988) Cancer Res 48(16), 4584-7. Third, we used this method to quantify the dynamics of drug distribution in ablated and non-ablated rabbit livers over time. The detailed experimental procedure of the fluorescence imaging method is described below.

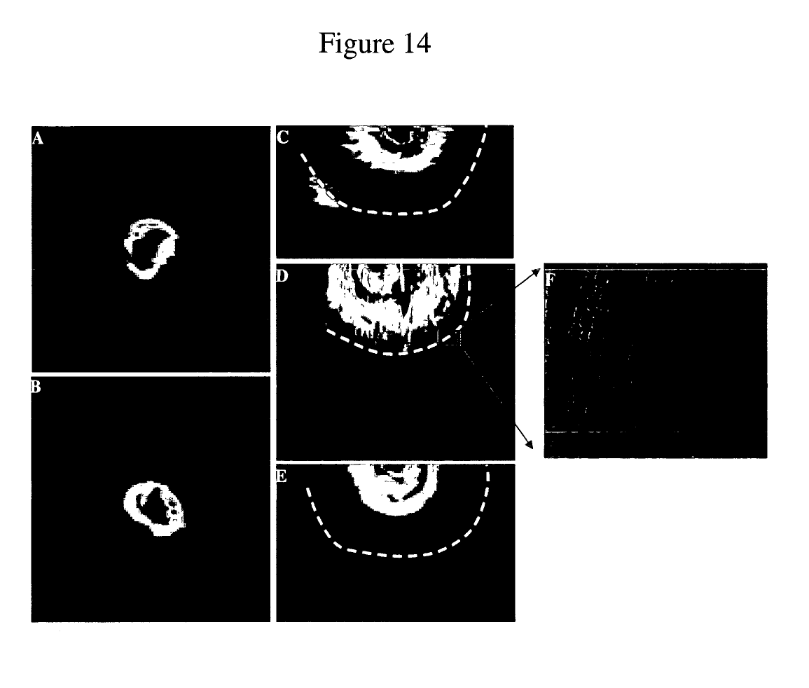

FIG. 14 compares the 2D doxorubicin distribution profiles between non-ablated and thermoablated liver tissues. In this set of experiments, a monolithic millirod with burst release kinetics (millirod (a) in FIG. 2) was implanted in either non-ablated or thermoablated livers. At different implantation times, the liver was harvested and the drug distribution profiles were obtained perpendicular to the long axis of the millirod. FIG. 14 displays the 2D distribution profiles as pseudo-color images constructed in MatLab 5.3. The red color in the images corresponds to a higher doxorubicin concentration. White dashed lines in the figures represent the ablated-normal tissue boundary, which was obtained by comparing the fluorescence image with an optical image of histology slides where this boundary is well-defined (see next section).

FIGS. 14A and 14B demonstrate that in normal livers, doxorubicin distribution is limited to the implantation site and almost no doxorubicin is detected 1 mm away from the millirod-tissue interface. We hypothesize that high perfusion in the non-ablated liver tissues resulted in the narrow distribution pattern, which is consistent with the observation by Dr. Saltzman and coworkers in the brain tissue. (Fung, L. K., Shin, M., Tyler, B., Brem, H. and Saltzman, W. M. (1996) Pharm Res 13(5), 671-82; Strasser, J. F., Fung, L. K., Eller, S., Grossman, S. A. and Saltzman, W. M. (1995) J Pharmacol Exp Ther 275(3), 1647-55). In comparison, the pattern of distribution is much larger in the ablated tissues (14C-E). We believe the larger distribution pattern is due to the destruction of liver vasculature by thermoablation (as evident from histology analysis herein). In this case, drug diffusion is the dominant transport process instead of drug washout by liver perfusion. Furthermore, the distribution patterns in the ablated tissues changed with time following millirod implantation. The distribution pattern at 24 hours (14D) is larger than those at 4 (14C) and 48 hours (14E). We speculate that the smaller distribution pattern at 48 hours (14E) is due to the depletion of doxorubicin from the millirod (in vitro release studies showed that all the drugs were released in ˜10 hours from this millirod, see FIG. 2).

In addition to fluorescence imaging study, we also used fluorescence microscopy to examine the liver slices. FIG. 14F shows the true fluorescence image at the boundary of ablated and non-ablated tissue. Since the two peak emission wavelengths of doxorubicin are 560 and 593 nm, the fluorescence image shows the red color. The more obvious color contrast between the ablated and non-ablated regions in FIG. 14F compared with 14D is due to the different color scales, which reflect a concentration range of 0-50 μg/g in 14F (true color) and 0-500 μg/g in 14D (assigned color). FIG. 14F demonstrates the feasibility of using fluorescence microscopy to evaluate the local distribution of doxorubicin in the liver tissue.

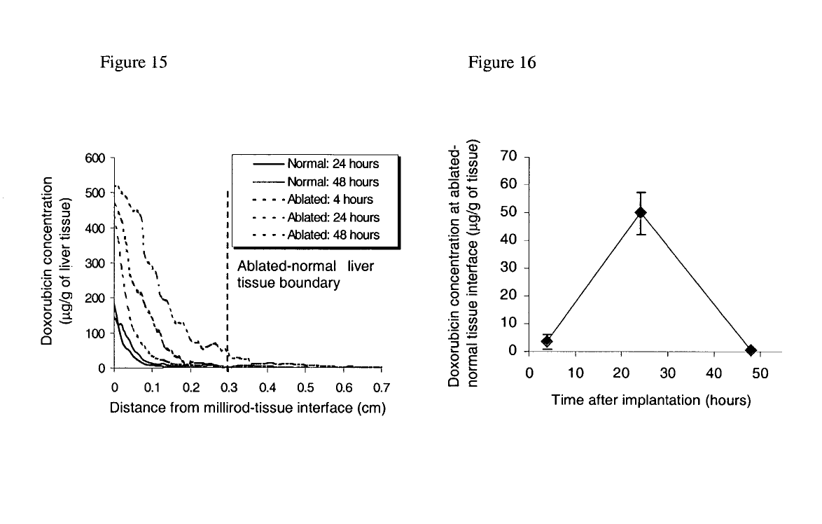

FIG. 15 provides a quantitative concentration vs. distance profile for different experimental conditions. Each profile was obtained by averaging 4 different radial profiles in a 2D image. Based on FIG. 15, we also obtained a Cₛ-t curve, which describes the doxorubicin concentration at the thermoablation boundary vs. implantation time (FIG. 16). Results show that at 24 hours, the value of Cₛ reached a maximum of 50 μg/g concentration, which is higher than the reported cytotoxic concentration of doxorubicin (6.4 μg/g). (Ridge, J. A., Collin, C., Bading, J. R., Hancock, C., Conti, P. S., Daly, J. M. and Raaf, J. H. (1988) Cancer Res 48(16), 4584-7.) The doxorubicin concentration drops below the therapeutic level after 48 hrs, demonstrating the need of sustained drug release to accompany the burst release to maintain the therapeutic drug level at the site of action.

### EXAMPLE 3

This example describes the design and development of a membrane-encased polymer millirod for the sustained release of an anticancer drug, 5-fluorouracil (5-FU). 5-FU is a commonly used drug for liver tumors (Kurokawa Y, Hasuike Y, Hattori T, Hayashi S, Fujitani K, Shin E, Mishima H, Sawamura T, Nishisho I, Kobayashi K, et al. (1999) Gan To Kagaku Ryoho, 26, 1737-40; Ekberg H, Tranberg K G, Persson B, Jeppsson B, Nilsson L G, Gustafson T, Andersson K E, Bengmark S. (1988) J Surg Oncol, 37, 94-9; Matsui K, Tomoe T, Terajima S, Yamasato M, Kondo J. (1970) Gan No Rinsho, 16, 43-7) and it is a suicide inhibitor to thymidylate synthase, a key enzyme involved in the conversion of dUMP to dTMP.

The millirod consists of two functional compartments: (1) an inner 5-FU-loaded monolithic millirod as the drug depot and (2) an outer NaCl-impregnated polymer membrane to control the release rate of 5-FU. The inner millirod is fabricated by a compression-heat molding procedure to permit the entrapment of 5-FU particles in the poly(D,L-lactide-co-glycolide) (PLGA) matrix. The drug loading density is controlled at 30 w/w % to achieve a burst release of 5-FU (>90% of the drug are released within 48 hours) from the monolithic millirod. The NaCl-impregnated PLGA membrane is generated by solvent casting and is then wrapped over the monolithic millirod to produce the membrane-encased millirod. Scanning electron microscopy shows that dissolution of NaCl particles produces a semi-permeable polymer membrane to provide a sustained release of 5-FU. The membrane thickness and the density of NaCl particles inside the membrane are useful parameters to control the release kinetics of 5-FU. Under the experimental conditions in this study, sustained release of 5-FU (rates between 0.1 and 0.4 mg/(day·cm of millirod)) is achieved for 2 to 5 weeks in phosphate buffered saline (pH 7.4) at 37° C. Results from this study demonstrate that membrane-encased polymer millirods provide controllable sustained-release kinetics for applications in intratumoral drug delivery.

### MATERIALS AND METHODS

Materials

Poly(D,L-lactide-co-glycolide) (lactide: glycolide=1:1, MW 50,000 Da, inherent viscosity 0.65 dL/g) was purchased from Birmingham Polymers, Inc. (Birmingham, Ala.). 5-Fluorouracil was purchased from Sigma (St. Louis, Mo.). Sodium chloride (NaCl), phosphate buffered saline (PBS) and methylene chloride were obtained from Fisher Scientific (Pittsburgh, Pa.). PLGA microspheres (size ˜5 μm) were produced by a single emulsion procedure. (Qian F, Szymanski A, Gao J. Fabrication and characterization of controlled release poly(D,L-lactide-co-glycolide) millirods, J. Biomed. Mater. Res., 55, 512-22 (2001))

Preparation of 5-FU-loaded, Monolithic PLGA Millirods

The monolithic millirods containing 10, 20 and 30 w/w % 5-FU were fabricated by a compression-heat molding procedure described previously. (Qian F, Szymanski A, Gao J. (2001) J. Biomed. Mater. Res., 55, 512-22.) Briefly, 5-FU powder and PLGA microspheres were weighed separately according to the final loading densities of 5-FU in the millirods. The two components were placed in a plastic tube and physically mixed by vortex for 10 minutes. The mixture was placed into a Teflon tube (ID 1.6 mm) and then the Telfon tube was placed inside a stainless steel mold. The mold was put inside an iso-temp oven at 90° C. (Fisher Model 282A, set point accuracy <2° C.) for two hours to allow the annealing of PLGA polymer. Compression pressure of 4.6 MPa was applied during the annealing process by copper weight. The monolithic millirods with 30 w/w % 5-FU were further used to fabricate the membrane-encased millirods.

Preparation of NaCl-Impregnated PLGA Films

A solvent casting method was used to prepare PLGA membranes containing NaCl particles. First, NaCl particles with size distribution between 90-150 μm were selected by sieves and the size of the particles was verified by SEM. The NaCi particles were then mixed together with PLGA polymer according to designed ratios and methylene chloride was added into the mixture. The volume of methylene chloride was measured so that the concentration of PLGA was 200 mg/mL. The suspension was vigorously vortexed to disperse NaCl particles homogenously through the viscous PLGA solution. The suspension was immediately poured into a Teflon dish (5 cm in diameter) and allowed to dry at room temperature for 48 hours and then under high vacuum for another 48 hours. After drying, the NaCl-impregnated PLGA film was peeled off the Teflon dish with forceps and the thickness of the membrane was measured by a micrometer at 10 different locations to calculate the average thickness. The membrane thickness was controlled by using different volumes of PLGA polymer suspension on the same Teflon dish.

Preparation of Membrane-Encased PLGA Millirods

Membrane-encased PLGA millirods were obtained by wrapping the monolithic millirods (30 w/w % 5-FU) with NaCl-impregnated PLGA films. The conjunction of the PLGA film was annealed by compression with a heated stainless-steel forceps. Both ends of the membrane-encased PLGA millirods were sealed by dipping the ends into 400 mg/mL PLGA solution in methylene chloride. The millirods were then dried for 24 hours in the air followed by another 24 hours under vacuum. The same procedure was repeated for millirods with different membrane structure and composition (Table 1).

SEM Analysis

Scanning electron microscopy (SEM, JEOL model 840) was used to study the morphology of the monolithic and membrane-encased PLGA millirods. Both the outer surface and the cross-section of the millirods were examined. Before SEM analysis, the sample was mounted on the aluminum stub by double-sided tape and sputter-coated with Pd (thickness 10 nm). SEM analysis was carried out at an accelerating voltage of 20 kV.

In vitro Release Study

The release study was carried out in PBS buffer (pH=7.4) at 37° C. Each millirod was placed in a glass vial containing 10 mL PBS buffer. The sample vials were placed in an orbital shaker (C24 model, New Brunswick Scientific) with a rotating speed of 100 RPM. At each time point, the solution was removed for UV measurement and 10 mL of fresh PBS solution was added. The concentration of released 5-FU in PBS buffer was determined at its maximum adsorption wavelength of 266.1 nm by a Hitachi U3210 UV-Vis spectrophotometer. The extinction coefficient of 5-FU at this wavelength was measured to be 46.1 mL/(cm·mg). The release study for monolithic millirods with 10, 20 and 30 w/w % 5-FU was carried out for 7 days, while for the membrane-encased millirods, release study continued until all of the 5-FU was released.

Characterization of Monolithic Millirods with Different Loading Density of 5-FU

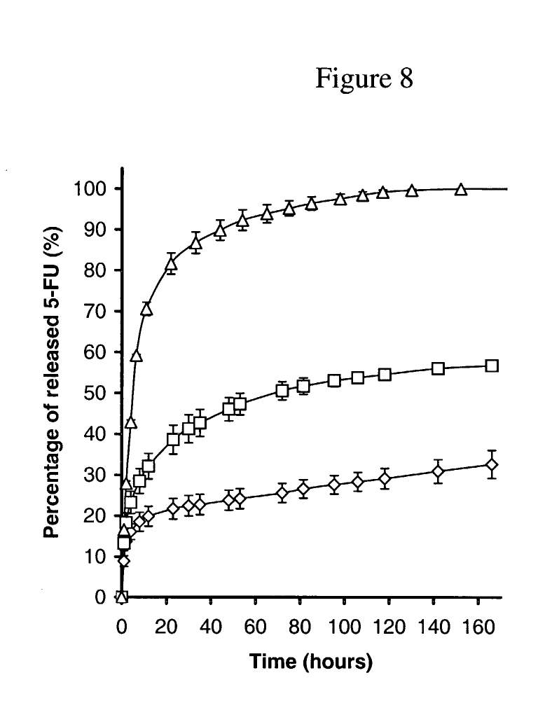

FIG. 8 illustrates the release profiles of monolithic millirods with 10, 20 and 30 w/w % loading density of 5-FU. All three compositions showed typical diffusion-based release kinetics at the early release phase (t<40 hours). At closer examination, millirods with different loading density of 5-FU showed different release percentage when reaching the slow release or plateau phase. For example, at 80 hours, almost 95% of the incorporated 5-FU was released from the 30 w/w % millirods while only 45% and 25% of 5-FU were released from the 20 w/w % and 10 w/w % millirods, respectively. In addition, the drug release rates decreased dramatically in the plateau phase compared to the initial phase for all the millirods despite significant amount of 5-FU still remained inside 10 w/w % and 20 w/w % millirods.

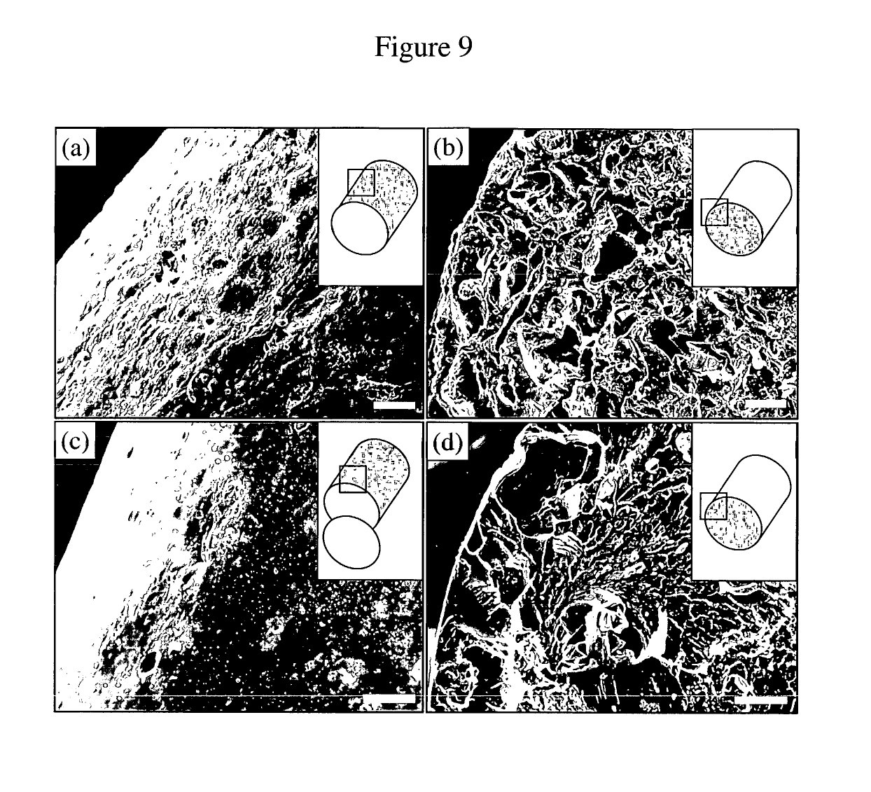

To understand the mechanism of 5-FU release from the millirods, we used SEM to characterize the microstructure of the 30 w/w % and 10 w/w % millirods. FIGS. 9a and 9b show the morphology of outer surface and cross-section of 30 w/w % 5-FU millirod after 2 days of release study in PBS buffer, respectively. At this time, more than 90% of the 5-FU was released from the PLGA millirod. The outer surface appears to be rough and contains holes as a result of dissolution of 5-FU particles at the millirod surface (FIG. 9a). Examination of the cross-section shows that dissolution of 5-FU particles led to the formation of empty interconnecting pores and channels (FIG. 9b) in the PLGA matrix. These results are consistent with the high percentage of 5-FU release (>90%) and indicate that 30 w/w % loading of 5-FU is sufficiently high to generate a continuous 5-FU phase inside the PLGA matrix.

FIGS. 9c and 9d show the morphology of outer surface and cross-section of 10 w/w % millirod after 2 days of release, respectively. The surface of 10 w/w % millirod (FIG. 9c) appears to be smoother and less porous than that of 30 w/w % millirod (FIG. 9a). Furthermore, no interconnecting channels were observed in the cross-section image. Empty pores induced by leaching of 5-FU particles were located closely to the surface of the millirod (FIG. 9d). These results are consistent with the release study in which majority of 5-FU (˜80%) still remained inside the 10 w/w % millirod after 2 days (FIG. 8).

Studies on the monolithic millirods (FIG. 8) demonstrate that varying the 5-FU loading density in the polymer matrix only provides limited control over the release kinetics of the drug. In all three conditions, burst release of 5-FU was observed in the first day followed by a plateau phase where the release rates were dramatically decreased in the following week. The observed release profiles are consistent with a percolation theory used in diffusion-controlled drug release systems. (Bonny J D, Leuenberger H (1991) Pharmaceutica Acta Helvetiae, 66, 160-4; J. D. Bonny H L (1993) Pharmaceutica Acta Helvetiae, 68, 25-33.) In this theory, a percolation threshold exists in a binary system consisting of drug and polymer matrix. The percolation threshold corresponds to a maximum drug loading density that ensures the formation of continuous drug phase inside the polymer matrix. Below this value the incorporated drug phase is isolated and surrounded by the insoluble polymer matrix, which leads to an incomplete release; above this value the drug phase forms interconnected channels and results in a complete release. Based on the release profiles of 10, 20 and 30 w/w % 5-FU millirods in FIG. 8, we infer that the percolation threshold of 5-FU/PLGA binary system is between 20-30 w/w %. This is supported by the SEM analysis where 30 w/w % millirods showed interconnected channels after 2 days of drug release (FIG. 9b) while in 10 w/w % millirods only drug particles with direct contact to the millirod surface were released (FIG. 9d).

There are multiple challenges that limit the use of monolithic millirods to control the release kinetics of 5-FU. First, sustained release of drugs over several weeks is difficult to achieve. After the initial burst release in the first 2 days, drug release reaches a plateau phase and little 5-FU is released in the following days (FIG. 8). Second, there are limited parameters in a monolithic device to control the release kinetics. Although drug loading density directly affects the release rates in the burst phase, it does not provide an accurate control of release rates in the plateau phase. Third, when the drug loading density is below the percolation threshold, “dose dumping” of the remaining 5-FU in the polymer matrix may occur as a result of bulk degradation behavior of PLGA. (Vert SLaM (1995) Chapman & Hall.) In this case, water-soluble excipient molecules (e.g., glucose) can be incorporated into the devices to increase matrix porosity for a complete release, however, the release kinetics will resemble those of 30 w/w % millirods instead of a sustained-release profile.

Surface Analysis of NaCl-Impregnated PLGA Membrane

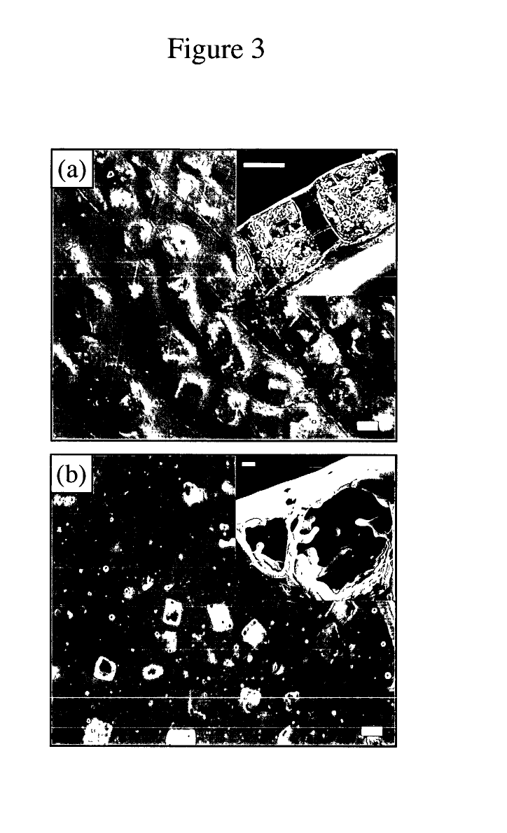

We used a solvent casting method to produce the NaCl-impregnated PLGA membrane. In this study, we fixed the size distribution of NaCl particles (90-150 μm) and varied two parameters in NaCl density and film thickness to control the membrane permeability. We used SEM to analyze the particle dispersion and pore formation in the PLGA film. FIG. 3a shows the surface and cross-section (inset) of 50 w/w % NaCl-impregnated PLGA membrane before hydration. The SEM analysis shows that NaCl particles were embedded inside the PLGA matrix and the dispersion of NaCl particles was homogenous. The thickness of the membrane was measured to be 137±18 μm. The cross-section image (FIG. 3a inset) shows that NaCl particles almost bridged the two opposite surfaces of the membrane, which is consistent with the size distribution of the NaCl particle (90-150 μm). FIG. 3b shows the surface and cross-section (inset) of the same membrane after 48 hours of hydration study in PBS buffer. The results clearly demonstrate that NaCl particles were leached out from the PLGA membrane, leaving empty pores across the membrane. The porous membrane became a semi-permeable barrier that can be used to control the release kinetics of drugs from a burst release device.

Release Study of Membrane-Encased Millirods

Table 1 lists five types of membrane-encased millirods with different membrane properties. In these membrane-encased devices, we chose 30 w/w % monolithic millirods as the inner millirods. As shown in FIG. 8, 30 w/w % millirods gave burst release kinetics where more than 90% of 5-FU was released in the first two days. PLGA membranes with different NaCl loading and membrane thickness were used to control the release rate from the polymer millirods. The NaCl loading density in the membrane varies from 10 to 50 w/w % and the membrane thickness from 137±18 to 215±20 μm (Table 1).

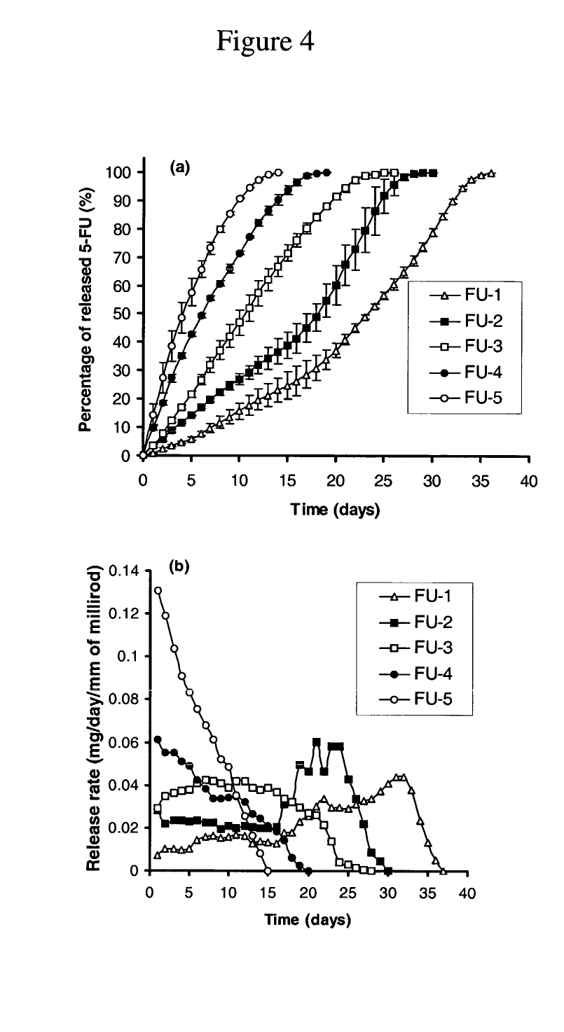

FIG. 4a shows the cumulative percentage of released 5-FU over time for different membrane-encased millirods. Compared to monolithic millirods, the membrane-encased millirods clearly demonstrate the sustained-release kinetics. For example, the time for the release of 50% 5-FU (t_{½}) is 5 hours for the 30 w/w % monolithic millirods (FIG. 8). In comparison, the values of t_{½} are 4, 6, 10, 18 and 24 days for FU-5, FU-4, FU-3, FU-2 and FU-1 millirods, respectively. Depending on the use of different membranes, release over a period 20 to 120 times longer was achieved compared to the monolithic device. Moreover, the sustained-release kinetics can be controlled by the membrane properties. In a series of control experiments, we discovered that increasing the loading density of NaCl in the membrane while maintaining approximately the same membrane thickness (e.g., from FU-1 to FU-4) led to decreased values of t_{½} and faster release kinetics. Meanwhile, increasing the membrane thickness while maintaining the same NaCl loading density (e.g., from FU-5 to FU-4) led to increased values of t_{½} and slower release kinetics (Table 1). Closer examination of the release curves also shows that the FU-1 and FU-2 millirods displayed two-phase release profiles where the release rates increased at approximately day 17.

To quantify the rate profiles of different membrane-encased millirods, we plotted the release rates of 5-FU over time (FIG. 4b). Results show that the drug release rates of FU-5 and FU-4 millirods kept decreasing over time. The release rate of FU-5 millirods was approximately 0.13 mg/(day·mm of millirod) at the beginning of the release study, and decreased to 0.05 mg/(day·mm of millirod) after 10 days when more than 90% of 5-FU was released. For FU-4 millirods, the initial release rate was approximately 0.06 mg/(day·mm of millirod) and the rate decreased to 0.025 mg/(day·mm of millirod) after 15 days when 90% 5-FU was released. In contrast, the release rate of FU-3 millirods was maintained in the range of 0.03 to 0.045 mg/(day·mm of millirod) in the first 20 days, and the device almost worked as a zero-order release device to deliver majority of the drug dosage (>90%).

Consistent with the observation in FIG. 4a, the rate profiles of FU-1 and FU-2 millrods displayed two distinguished phases of drug release. Before day 17, both millirods behave similarly to a zero-order release device. In this earlier phase, the release rates of FU-1 and FU-2 millirods were 0.010-0.016 and 0.020-0.025 mg/(day·mm of millirod), respectively. However, the release rates of both types of millirods increased in the later phase before 90% of the drug dosage was released. More specifically, the release rates of FU-2 millirods were elevated from 0.020 at day 16 to 0.060 mg/(day·mm of millirod) at day 21. Similarly, the release rates of FU-1 millirods increased continuously from 0.012 at day 16 to 0.045 mg/(day·mm of millirod) at day 32.

SEM Analysis of FU-2 Millirods

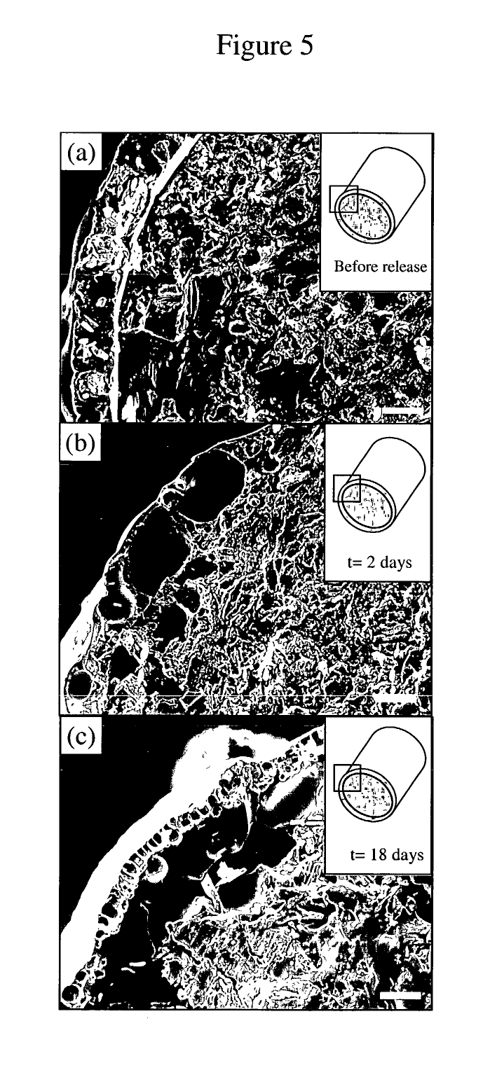

To gain insight on the two-phase release kinetics, we used SEM to analyze the microstructure of the FU-2 millirods at different times during the release study. FIG. 5 shows the cross-sections of the FU-2 millirods before release, 2 and 18 days after release in the PBS buffer. FIG. 5a demonstrates the two-compartment structure of the membrane-encased millirods: the NaCl-impregnated outer membrane and the inner monolithic millirod. SEM image after 2 days of release study (FIG. 5b) shows that the NaCl particles were leached from the outer membrane and the membrane became porous. A small portion of 5-FU that was close to the membrane was also released. However, the extent of release was significantly smaller than that of the 30 w/w % monolithic millirod after the same time period (FIG. 5b). This is consistent with the release data that only 10% of 5-FU was released from the FU-2 millirod while over 90% was released from the monolithic millirod after 2 days. After 18 days of release, SEM analysis shows obvious signs of polymer degradation in the outer membrane (FIG. 5c). Small pores were uniformly observed at the outer surface of the PLGA membrane. Since the size of these pores (10-20 μm in diameter) is significantly smaller than the NaCl particles, we believe that they are the result of polymer degradation and dissolution, which is consistent with the degradation studies of PLGA films reported by Mikos. (Lu L, Garcia C A, Mikos A G (1999) J. Biomed. Mater. Res., 46, 236-44 (1999); Lu L, Peter S J, Lyman M D, Lai H L, Leite S M, Tamada J A, Uyama S, Vacanti J P, Langer R, Mikos A G (2000) Biomaterials, 21, 1837-45.) For FU-2 millirods, formation of micropores leads to an increase in membrane permeability as well as the release rate in the second release phase as observed in FIG. 4b.

Here we report the design and development of a novel membrane-encased polymer millirod to sustain the release of 5-FU for 2-5 weeks. This device consists of two modular components: a monolithic millirod that supplies 5-FU based on a predetermined drug dosage, and a polymer membrane that controls the release rates. In the membrane-encased millirod, a monolithic millirod with a burst and complete release of 5-FU is necessary as the drug depot. Here we chose the 30 w/w % monolithic millirod (>90% 5-FU were released in less than 2 days, FIG. 8) in the proof-of-principle studies. Under the circumstance when the drug dosage is below the percolation threshold, an excipient molecule such as NaCl or glucose can be introduced to achieve the burst and complete release kinetics from the monolithic millirod.

Results from this study demonstrate that membrane-encased millirods are much more versatile and effective to control the drug release kinetics than the monolithic millirods. In a series of experiments (FIG. 4a), we showed that a sustained release of 5-FU has been achieved from 2 (FU-5 millirod) to 5 weeks (FU-1 millirod). Moreover, the duration and rate of drug release can be controlled by varying the permeation properties of the PLGA membrane. In this study, we controlled the membrane permeability by varying the membrane thickness and porosity (NaCl loading density) (Table 1). Thinner membrane and higher NaCl loading lead to faster release of 5-FU. Drug release rate controls the amount of drug released into the tumor tissue per unit time, which subsequently dictates the drug concentration distribution profiles in the ablated tissue. Depending on the ablation size and drug transport properties in the normal and ablated tissues, an optimal drug release rate exists to permit the reaching of drug concentration at the ablation boundary to the therapeutic level.

In certain embodiments, a slower degrading polymer (e.g., poly(L-lactic acid), with a half-weight degradation time of 10-40 weeks depending on molecular weight) (Lu L, Peter S J, Lyman M D, Lai H L, Leite S M, Tamada J A, Vacanti J P, Langer R, Mikos AG (2000) Biomaterials, 21, 1595-605) can be used to replace PLGA (3 weeks) in order to slow degradation and concomitant increased permeability of the membrane. (Lu L, Peter S J, Lyman M D, Lai H L, Leite S M, Tamada J A, Uyama S, Vacanti J P, Langer R, Mikos A G (2000) Biomaterials, 21, 1837-45). In faster release systems (such as millirods FU-4 and FU-5), a water-soluble polymer (e.g., dextran) can be blended inside the monolithic millirods to facilitate the dissolution and diffusion of the drug contained in the core. Third, in addition to NaCl particles, other pore-forming materials can also be used to control the membrane permeability. NaCl particles are simple and inexpensive materials that permit easy control over the porosity and pore size in the PLGA membrane. In applications where high ionic concentrations are not desirable, other organic-based materials (e.g., glucose particles, polyethylene glycol polymer) can be used to control the membrane permeability and the drug release rate from the membrane-encased millirods.

TABLE 1
Structural composition and release
properties of different membrane-encased millirods.

| Millirod | Inner millirod | Outer membrane | Membrane | t_{½} |
|---|---|---|---|---|
| code | 5-FU % (w/w) | NaCl % (w/w) | thickness (μm)^{a} | (days)^{b} |
| FU-1 | 30 | 10 | 209 ± 13 | 24 |
| FU-2 | 30 | 20 | 191 ± 14 | 18 |
| FU-3 | 30 | 30 | 206 ± 17 | 10 |
| FU-4 | 30 | 50 | 215 ± 20 | 6.0 |
| FU-5 | 30 | 50 | 137 ± 18 | 4.1 |

^{a}The standard deviation was obtained from 10 measurements.
^{b}t_{½}corresponds to the time when 50% 5-FU is released.

### EXAMPLE 4

This example describes a three-layer device that permits dual-release kinetics for release of doxorubicin. The three-layer polymer millirod is produced by a dip-coating method, and in vitro studies demonstrate dual-release kinetics in which a burst release occurs within 2 hours followed by sustained release over 7 to 10 days. Independent control of the burst and sustained release rates is achieved by varying the structural composition of the outer and middle layers of the millirods, respectively. Results from this study provide the rational basis and experimental feasibility of dual-release millirods for further efficacy studies in solid tumors.

Materials

Poly(D,L-lactide) (PLA, inherent viscosity 0.67 dL/g) and poly(D,L-lactide-co-glycolide) (PLGA, lactide: glycolide=1:1, MW 50,000 Da, inherent viscosity 0.65 dL/g) were purchased from Birmingham Polymers, Inc. (Birmingham, Ala.). Poly(ethylene glycol) (PEG, Mₙ 4,600) and poly(ethylene oxide) (PEO, Mᵥ 200,000) were obtained from Aldrich (Milwaukee, Wis.). Doxorubicin HCl solution was purchased from Bedford Laboratories (Bedford, Ohio).

Fabrication of Doxorubicin Loaded, Dual-Release Millirods

The doxorubicin HCl solution was first desalted by dialysis in distilled water and then the purified doxorubicin solution was lyophilized to provide fine powder. PLGA microspheres (size: 5 μm) were produced by a single emulsion procedure. (F. Qian, A. Szymanski, J. Gao (2001) J Biomed Mater Res, 55, 512-522.) Monolithic PLGA millirods containing 16% doxorubicin, 24% NaCl and 60% PLGA were fabricated by a compression-heat molding procedure. (F. Qian, A. Szymanski, J. Gao (2001) J Biomed Mater Res, 55, 512-522.) Briefly, doxorubicin, NaCl and PLGA microspheres were weighed separately according to the final loading densities and the three components were placed in a plastic tube and physically mixed by vortex for 10 minutes. The mixture was placed into a Teflon tube (ID 1.6 mm) and then the Telfon tube was placed inside a stainless steel mold. The mold was put inside an iso-temp oven at 90° C. (Fisher Model 282A, set point accuracy <2° C.) for two hours to allow the annealing of PLGA polymer. Compression pressure of 4.6 MPa was applied during the annealing process by copper weight. After cooling down to room temperature, the millirods were pushed out of the Teflon tube by a stainless-steel plunger. The monolithic millirods have a diameter of 1.6 mm, and their length was cut to 10 mm.

Dual-release millirods were fabricated by applying two additional dip-coating procedures on the monolithic millirods. The PEG/PLA layer (middle layer) was formed by dipping the monolithic PLGA millirods into PEG/PLA solution in CH₂Cl₂. The total polymer concentration was 200 mg/mL and three different PEG in PLA percentages were used: 5%, 10% and 20%. The dipping speed was controlled by a vertically placed syringe pump at 2 mm/sec. After the control layer was completely dried, the burst layer was formed by dipping the millirod into doxorubicin/PEO suspension (100 mg/mL, 75% doxorubicin, 25% PEO in CH₂Cl₂). High molecular weight PEO was used to increase the viscosity of the dipping solution. The number of dips in doxorubicin/PEO suspension was used to control the burst dose. The dimension of the millirods is 10 mm in length, 1.8-2.0 mm in diameter depending on the thickness of the coated layers.

In vitro Release Studies

In vitro release studies were carried out in 25 mM Tris buffer at 37° C. Each millirod was placed in a glass vial containing 2 mL Tris buffer. The sample vials were placed in an orbital shaker (C24 model, New Brunswick Scientific) with a rotating speed of 100 RPM. At each time point, 2 mL of solution were removed for concentration measurement and 2 mL of fresh buffer were added. The concentration of released doxorubicin was measured by a UV-Vis spectrophotometer (Perkin-Elmer Lambda 20 model) at its maximum adsorption wavelength (480.8 nm). The extinction coefficient of doxorubicin at this wavelength is 16.8 mL/(cm.mg).

SEM Analysis

Scanning electron microscopy (SEM, JEOL model 840) was used to study the morphology of the cross-section of the dual-release PLGA millirod. Freeze-fracturing in liquid nitrogen was used to provide a smooth and even millirod cross-section. Before SEM analysis, the sample was mounted on an aluminum stub by double-sided tape and sputter coated with Pd (10 nm thick). SEM analysis was carried out at an accelerating voltage of 20 kV.

Structural Composition of Dual-Release Millirods

The dual-release polymer millirods consist of three structural components. The outer water-soluble doxorubicin/PEO layer provides the initial burst release of the drug after contact with biological fluid. After dissolution of the outer layer, the sustained release rate of doxorubicin is controlled by the middle PEG/PLA layer. The inner core of doxorubicin-PLGA matrix serves as the drug reservoir for sustained release. For this study, the inner monolithic PLGA millirods (16% doxorubicin) were kept the same for all the dual-release millirods. The PEG composition in the middle layer (5, 10, 20%) was varied to control the sustained release rate. Higher PEG content produces higher porosity in the middle layer and, therefore, a faster drug permeation and release rate. The burst dose of doxorubicin was controlled by the thickness of the outer PEO layer loaded with 75% doxorubicin. Table 2 lists five types of dual-release millirods with different structural compositions and release properties.

In vitro Characterization of Millirod Release Profiles

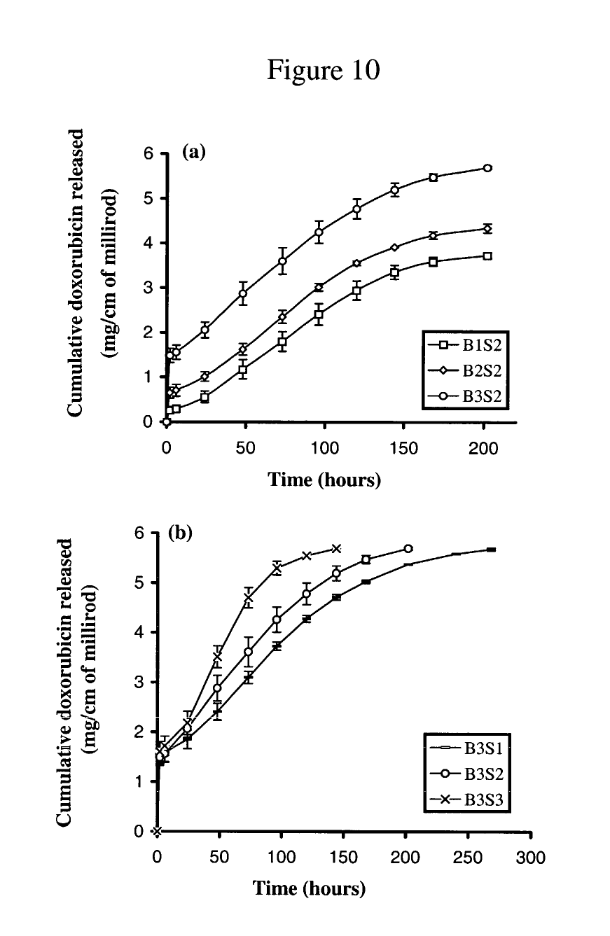

FIG. 10a shows the cumulative release of doxorubicin from three types of dual-release millirods with different burst doses (A_{B}), but similar sustained-release rates (R_{D}). These millirods shared the same inner core and rate-control (middle PEG/PLA) layer, but the thickness of the outer layer differed. All three types of millirods demonstrated the dual-release kinetics: a steep initial burst release phase due to the dissolution of the outer PEO layer followed by a sustained release phase controlled by the middle layer (10% PEG in PLA). Millirods B1S2, B2S2 and B3S2 have burst doses of 0.26, 0.65 and 1.49 mg/(cm millirod), respectively, within the first 2 hours of release. For these millirods, the sustained dose (A_{M}, amount of doxorubicin contained inside the inner drug reservoir) was approximately 3.5 mg/(cm millirod), and the sustained release (constant slope) phase was maintained for about 7 days (or 170 hours) at approximately 0.4 mg/(day·cm millirod).

FIG. 10b shows the cumulative release of doxorubicin from three types of dual-release millirods with the same burst dose, but different sustained release rates. These millirods shared the same inner core and burst dose layer (outer PEO layer), but the rate-control (middle PEG/PLA) layers differed. The PEG composition in the middle layer was set at 5, 10 and 20% for B3S1, B3S2 and B3S3 millirods, respectively. The cumulative release profiles demonstrate similar burst dose at 1.55 mg/(cm millirod), but the sustained release rates increased with the increase of PEG composition. The average release rates were 0.27, 0.43 and 0.60 mg/(day·cm of millirod) for B3S1, B3S2 and B3S3 millirods, respectively. Since all the millirods shared the same sustained dose (A_{M}), the different release rates led to different time duration for the three types of dual-release millirods (FIG. 10b).

SEM Study of Millirod Microstructure

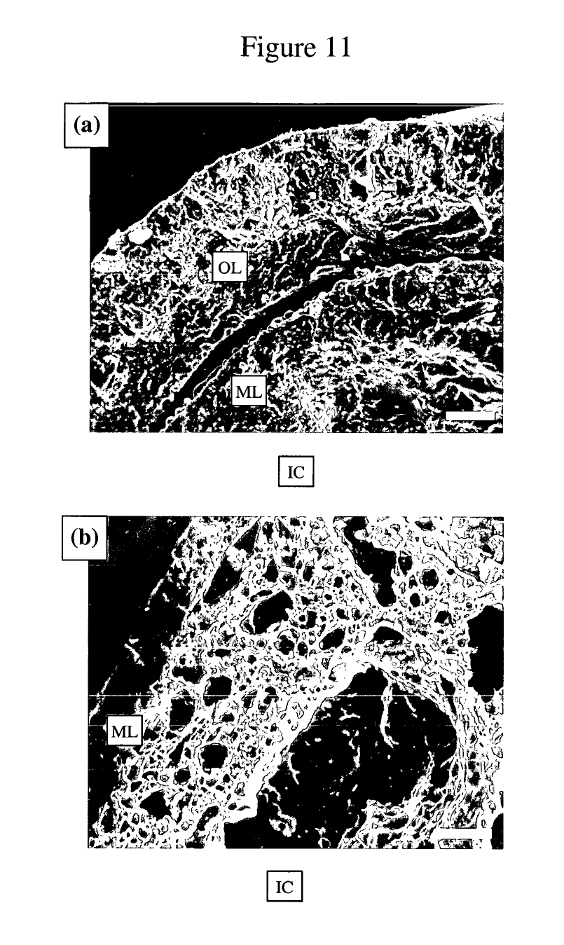

To obtain mechanistic insight on the dual-release kinetics, we used SEM to analyze the microstructure of the doxorubicin millirods before and after the release experiments. FIG. 11a shows the SEM image of the cross-section of a representative B3S2 millirod before release. It clearly demonstrates the three-layer structure of the dual-release millirod: the outer doxorubicin/PEO layer, the middle PEG/PLA layer, and the inner monolithic millirod. The thickness of the outer and middle layers is approximately 150 and 80 μm, respectively. SEM analysis (FIG. 11b) of the same millirod composition after 7 days of drug release in Tris buffer (pH 7.4) shows that the outer PEO layer, which produces the initial burst, was completely dissolved. The PEG/PLA layer appeared to be porous, probably due to the leaching of water-soluble PEG molecules from the hydrophobic PLA matrix. The resulting membrane became the semi-permeable barrier that controls the rate of release from the inner drug reservoir. Finally, SEM shows the interconnecting pores and channels inside the polymer matrix of the inner core, which is consistent with the complete release of doxorubicin and NaCl (FIG. 10).

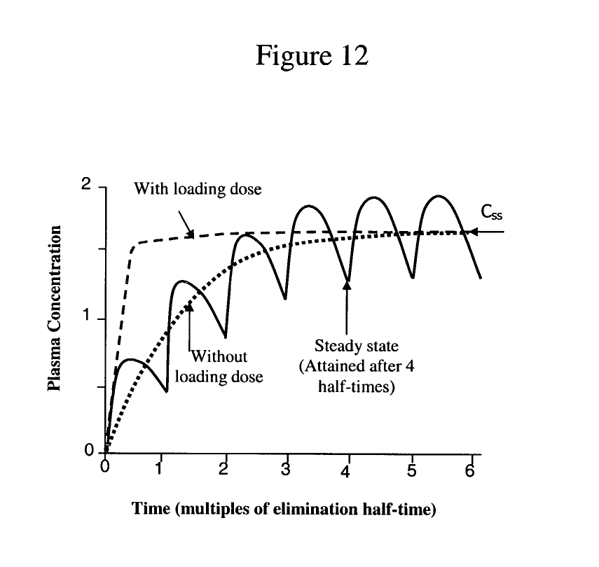

The delivery of a therapeutic agent to the site of action is the defining objective of any pharmaceutical treatment. The accessible concentration of the drug at the site of action and tissue exposure time are directly related to the pharmacological responses, whether therapeutic or toxic. In systemic chemotherapy, drug plasma concentrations (ideally, drug concentrations at the site of action should be used) are usually measured over time, and the area under the concentration-time curve (AUC) is calculated and related to pharmacological response. Typically, a desired steady-state concentration (Cₛₛ) of the drug is chosen and a desirable range for the Cₛₛ is defined as the therapeutic range. (L. Z. Benet, D. L. Kroetz, L. B. Sheiner (1996) Goodman & Gilman's The Pharmacological Basis of Therapeutic, ed. 9th. New York: McGraw-Hill Health Professions Division, 3-27.) Because most anticancer drugs have narrow therapeutic indices, their clinical applications require careful design of dosage regimens to achieve a fine balance between efficacy and toxicity. FIG. 12 illustrates the concentration-time curves for drugs either administered in a series of repeated doses or as a continuous infusion in systemic chemotherapy. The figure shows that continuous infusion of drugs permits a significantly less variable concentration range at Cₛₛ than intermittent dosing. In addition, the use of a loading dose permits a much faster attaining of Cₛₛ than the otherwise continuous dosing rate. The combined dosage administration and the resulting “immediate and sustained” effect in systemic chemotherapy provide the conceptual basis for the design of polymer devices in our drug delivery applications.

The first step in a rational and quantitative design of polymer millirods that can deliver anticancer drug by an “immediate and sustained” way requires the development of an appropriate mathematical model. For specific application in a thermally ablated liver tumor, we developed a dynamic model that describes the drug transport process in ablated and non-ablated regions. The objective of local drug therapy is to deliver drug at a sufficiently high concentration to the boundary of ablated tissue to kill the residual cancer cells. This requires that the therapeutic drug can be delivered to the targeted region quickly and the drug concentration can be maintained for a prolonged time. In this paper, we present a framework of procedures and working curves by which to calculate parameters required for fabrication of a polymer drug-delivery system.

Model simulations with a dual-release polymer millirod indicate how an initial burst dose followed by sustained release can provide optimal delivery of an anticancer drug to the ablation boundary. Without a burst dose, it would take a zero-order release device many days to reach the targeted region at the therapeutic concentration. Conceptually, the burst dose and the sustained release rate can be “custom-designed” to meet the requirements to deliver therapeutic drug concentrations for differently sized tumors.

Based on the current model simulations, we developed doxorubicin-containing polymer millirods with a controllable burst dose and sustained-release rate. The three-layer design was partly derived from our previous design of membrane-encased, sustained-release millirods. (F. Qian, N. Nasongkla, J. Gao (2002) J Biomed Mater Res, 61, 203-211.) Several factors can be used to adjust the membrane permeability and sustained-release rate, such as the percentage of water-soluble components in the membrane, membrane thickness and tortuosity. (F. Qian, N. Nasongkla, J. Gao (2002) J Biomed Mater Res, 61, 203-211.) In the current study, PEG percentage in PLA provided effective controls over the release rates. The loading dose is introduced by an additional layer of doxorubicin/PEO, which can be easily controlled by the thickness of the layer and drug loading percentage. In vitro release studies (FIG. 10) demonstrated both the burst dose and the sustained release rate of the dual-release millirods are independently adjustable by design.

TABLE 2
Structural composition and release properties
of different types of dual-release millirods.

| | | Middle | Dip-coating | | |
|---|---|---|---|---|---|
| Millirod | Inner core | layer | times of the | A_{B}: | R_{D}: mg/ |
| code | doxorubicin % | PEG % | outer layer | mg/cm | (day · cm) |
| B1S2 | 16 | 10 | 1 | 0.25 | 0.37 |
| B2S2 | 16 | 10 | 2 | 0.65 | 0.39 |
| B3S2 | 16 | 10 | 3 | 1.48 | 0.43 |
| B3S1 | 16 | 5 | 3 | 1.55 | 0.27 |
| B3S3 | 16 | 20 | 3 | 1.60 | 0.60 |

References

All publications and patents mentioned herein, are hereby incorporated by reference in their entirety as if each individual publication or patent was specifically and individually indicated to be incorporated by reference.

Equivalents

Those skilled in the art will recognize, or be able to ascertain using no more than routine experimentation, many equivalents to the specific embodiments of the invention described herein. Such equivalents are intended to be encompassed by the following claims.

We claim:

1. A biodegradable dual-release drug delivery device, comprising:
a) a central core comprising a first bioactive agent, and
b) a first layer disposed around the central core comprising a second bioactive agent and a biodegradable polymer,
whereby, upon placement in a biological environment, the second bioactive agent is released, thereby resulting in sustained release of the first bioactive agent.

2. The device of claim 1, wherein release of the second bioactive agent renders the first layer porous.

3. The device of claim 1, wherein the first layer further comprises a water-soluble component that dissolves upon placement in a biological environment, thereby rendering the first layer porous.

4. The device of claim 1, further comprising a water-soluble second layer disposed around the first layer.

5. The device of claim 4, wherein the second layer comprises a third bioactive agent, which may be the same or different from the first and second bioactive agents.

6. The device of claim 4, wherein the second layer comprises a water-soluble polymer.

7. The device of claim 1, wherein the first and second bioactive agent are the same.

8. The device of claim 1, wherein the device is a cylindrical millirod.

9. The device of claim 1, wherein at least one of the first and second bioactive agents is an anti-cancer agent.

10. The device of claim 1, wherein the water-soluble component comprises a water-soluble polymer.

11. The device of claim 1, wherein the core comprises an excipient.

12. The device of claim 1, wherein the water-soluble component comprises water-soluble inclusions.

13. The device of claim 10, wherein the water-soluble component comprises crystals of a biocompatible salt or sugar.

14. The device of claim 1, wherein the device provides local delivery of the bioactive agent(s).

15. The device of claim 1, wherein the first layer comprises a polymer selected from polylactic acid (PLA) and poly(lactic-glycolic acid) (PLGA).

16. The device of claim 1, wherein the bioactive agent(s) comprise an analgesic agent, anti-cancer agent, antiinflammatory agent, anti-fungal agent, anti-viral agent, cell transport/mobility impending agent, beta-blocker, immunological response modifier, peptide or protein, heat shock protein, steroidal compound, neuroprotectant, antibiotic, antibacterial, antiallergenic, anti-inflammatory, decongestant, miotic and anti-cholinesterase, angiogenesis inhibitor, permeability enhancer, or mydriatic.

17. The device of claim 1, wherein the first bioactive agent is released at a therapeutically effective concentration for at least two days.

18. The device of claim 17, wherein the first bioactive agent is released at a therapeutically effective concentration for at least a week.

19. A method of administering a bioactive agent to a patient, comprising implanting into a patient a device of claim 1.

20. A biodegradable drug delivery device, comprising:
a) a central core comprising a first bioactive agent,
b) a first layer disposed around the central core, comprising a water-soluble component and a biodegradable polymer, and
c) a water-soluble second layer comprising a second bioactive agent disposed around the first layer,
whereby, upon placement in a biological environment, the second bioactive agent is released and the water-soluble component dissolves rendering the first layer porous, thereby resulting in sustained release of the first bioactive agent.

21. The device of claim 20, wherein the second layer comprises a water-soluble polymer.

22. The device of claim 20, wherein the first and second bioactive agent are the same.

23. The device of claim 20, wherein the device is a cylindrical millirod.

24. The device of claim 20, wherein at least one of the first and second bioactive agents is an anti-cancer agent.

25. The device of claim 20, wherein the water-soluble component comprises a water-soluble polymer.

26. The device of claim 20, wherein the core comprises an excipient.

27. The device of claim 20, wherein the water-soluble component comprises water-soluble inclusions.

28. The device of claim 27, wherein the water-soluble component comprises crystals of a biocompatible salt or sugar.

29. The device of claim 20, wherein the device provides local delivery of the bioactive agent(s).

30. The device of claim 20, wherein the first layer comprises a polymer selected from polylactic acid (PLA) and poly(lactic-glycolic acid) (PLGA).

31. The device of claim 20, wherein the bioactive agent(s) comprise an analgesic agent, anti-cancer agent, antiinflammatory agent, anti-fungal agent, anti-viral agent, cell transport/mobility impending agent, beta-blocker, immunological response modifier, peptide or protein, heat shock protein, steroidal compound, neuroprotectant, antibiotic, antibacterial, antiallergenic, anti-inflammatory, decongestant, miotic and anti-cholinesterase, angiogenesis inhibitor, permeability enhancer, or mydriatic.

32. The device of claim 20, wherein the first bioactive agent is released at a therapeutically effective concentration for at least two days.

33. The device of claim 33, wherein the first bioactive agent is released at a therapeutically effective concentration for at least a week.

34. A method of administering a bioactive agent to a patient, comprising implanting into a patient a device of claim 21.

35. The method of claim 19 or 34, wherein the device comprises an anticancer agent and is placed at the site of a thermoablated tumor.

36. The method of claim 19 or 34, wherein the device comprises an analgesic agent and is placed at the site of a wound.

37. The method of claim 19 or 34, wherein the device comprises an antibiotic or antifungal agent and is placed at the site of an infection.

38. A method for manufacturing a biodegradable drug delivery device, comprising:
a) providing a central core comprising a first bioactive agent, and
b) disposing a layer around the central core, the layer comprising a second bioactive agent, a water-soluble component, and a biodegradable polymer.

39. A method for manufacturing a biodegradable drug delivery device, comprising:
a) providing a central core comprising a first bioactive agent and an excipient,
b) disposing around the central core a first layer comprising a water-soluble component and a biodegradable polymer, and
c) disposing around the first layer a water-soluble second layer comprising a second bioactive agent.
